(19) United States
(12) Patent Application Publication
(10) Pub. No.: US 2020/0017586 A1
(43) Pub. Date: Jan. 16, 2020

(54) USE OF IMMUNE CHECKPOINT MODULATORS IN COMBINATION WITH ANTIGEN-SPECIFIC T CELLS IN ADOPTIVE IMMUNOTHERAPY

(71) Applicant: Memorial Sloan Kettering Cancer Center, New York, NY (US)

(72) Inventors: Richard John O'Reilly, Roxbury, CT (US); Susan Elizabeth Prockop, New York, NY (US); Ekaterina Doubrovina, Bronx, NY (US); Parastoo Bahrami Dahi, New York, NY (US)

(73) Assignee: Memorial Sloan Kettering Cancer Center, New York, NY (US)

(21) Appl. No.: 16/483,880

(22) PCT Filed: Feb. 6, 2018
(86) PCT No.: PCT/US18/16995

§ 371 Date: Aug. 6, 2019

Related U.S. Application Data
(60) Provisional application No. 62/455,860, filed on Feb. 7, 2017.

(51) Int. Cl. C07K 16/28; A61K 35/17; A61P 31/12

(52) U.S. Cl. CPC C07K 16/2818; A61P 31/12; A61K 35/17

(57) ABSTRACT

Provided herein are methods of treating a human patient, comprising administering to the human patient an inhibitory immune checkpoint inhibitor or stimulatory immune checkpoint activator and administering to the human patient a population of human cells comprising antigen-specific T cells that are derived from a T cell line restricted by a subdominant HLA allele or HLA allele combination. Also provided are methods of selecting such a T cell line and methods of selecting a T cell donor from whom to derive such a T cell line, for therapeutic administration to a human patient in combination with administration of an inhibitory immune checkpoint inhibitor or stimulatory immune checkpoint activator. Also provided are pharmaceutical compositions comprising an inhibitory immune checkpoint inhibitor or stimulatory immune checkpoint activator and a population of human cells comprising antigen-specific T cells that are derived from a T cell line restricted by a subdominant HLA allele or HLA allele combination.

### CROSS-REFERENCE TO RELATED APPLICATIONS

This application claims the benefit of U.S. Provisional Patent Application No. 62/455,860, filed Feb. 7, 2017, which is incorporated by reference herein in its entirety.

### 1. FIELD

Provided herein are methods of treating a human patient, comprising administering to the human patient an inhibitory immune checkpoint inhibitor or stimulatory immune checkpoint activator and administering to the human patient a population of human cells comprising antigen-specific T cells that are derived from a T cell line restricted by a subdominant HLA allele or HLA allele combination. Also provided are methods of selecting such a T cell line and methods of selecting a T cell donor from whom to derive such a T cell line, for therapeutic administration to a human patient in combination with administration of an inhibitory immune checkpoint inhibitor or stimulatory immune checkpoint activator. Also provided are pharmaceutical compositions comprising an inhibitory immune checkpoint inhibitor or stimulatory immune checkpoint activator and a population of human cells comprising antigen-specific T cells that are derived from a T cell line restricted by a subdominant HLA allele or HLA allele combination.

### 2. BACKGROUND

Antigen-specific T cells can be used in adoptive immunotherapy to treat infections and cancer, such as cytomegalovirus (CMV) infections, Epstein-Barr virus-associated lymphoproliferative disorder (EBV-LPD), and WT1 (Wilms Tumor 1)-positive leukemia and multiple myeloma (see, e.g., Koehne et al., 2015, Blood 126:98; Koehne et al., 2015, Biol Blood Marrow Transplant 21:1663-1678; O'Reilly et al., 2012, Seminars in Immunology 22:162-172; and Doubrovina et al., 2012, Blood 119:2644-2656).

Evaluation of T-cell responses against CMV has led to the identification of several immunodominant epitopes within the most immunogenic proteins of this virus, namely CMVpp65 and IE1, and their presenting HLA alleles. It was found that the HLA alleles presenting immunodominant epitopes exist in a hierarchical order within individuals co-inheriting specific haplotypes, and thus certain HLA alleles are consistently the alleles restricting the immunodominant T-cell response when co-inherited with other HLA alleles (see International Patent Application Publication No. WO 2016/073550). The hierarchy of HLA alleles presenting immunodominant epitopes is exclusively based on their level of functional activity. Interestingly, there is no correlation between the affinity of the binding of epitope to HLA protein and their capacity to elicit immunodominant T cell responses (see International Patent Application Publication No. WO 2016/073550). Agents that can augment the efficacy of antigen-specific T cells restricted by HLA alleles associated with subdominant T-cell responses have not been explored.

Citation of a reference herein shall not be construed as an admission that such is prior art to the present disclosure.

### 3. SUMMARY OF THE INVENTION

In one aspect, provided herein is a method of treating a human patient having or suspected of having a pathogen or cancer, comprising: (1) administering to the human patient an inhibitory immune checkpoint inhibitor; and (2) administering to the human patient a population of human cells comprising antigen-specific T cells that are specific for an antigen of the pathogen or cancer and are derived from a T cell line restricted by a first HLA allele or HLA allele combination expressed by the diseased cells in the human patient; wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

In specific embodiments, the inhibitory immune checkpoint inhibitor is an inhibitor of Programmed Cell Death 1 (PD1), Programmed Death Ligand 1 (PD-L1), Programmed Death Ligand 1 (PD-L2), Cytotoxic T-Lymphocyte Associated Protein 4 (CTLA4), Lymphocyte Activating 3 (LAG3), T-Cell Immunoglobulin And Mucin Domain-Containing Protein 3 (TIM3), V-Domain Ig Suppressor Of T Cell Activation (VISTA), Adenosine A2a Receptor (A2aR), B7 Homolog 3 (B7-H3), B7 Homolog 4 (B7-H4), B and T lymphocyte associated (BTLA), Indoleamine 2,3-Dioxygenase (IDO), Tryptophan 2,3-Dioxygenase (TDO), or Killer-Cell Immunoglobulin-Like Receptor (KIR).

In specific embodiments, the inhibitory immune checkpoint inhibitor is an antibody that binds to and inhibits the activity of the inhibitory immune checkpoint. In a specific embodiment, the antibody is a monoclonal antibody.

In specific embodiments, the inhibitory immune checkpoint inhibitor is an inhibitor of PD1. In a specific embodiment, the inhibitory immune checkpoint inhibitor is a monoclonal antibody that binds to and inhibits the activity of PD1. In a particular embodiment, the monoclonal antibody is nivolumab, pidilizumab, MEDI0680, or pembrolizumab.

In specific embodiments, the inhibitory immune checkpoint inhibitor is an inhibitor of PD-L1. In a specific embodiment, the inhibitory immune checkpoint inhibitor is a monoclonal antibody that binds to and inhibits the activity of PD-L1. In a particular embodiment, the monoclonal antibody is mpd13280A, durvalumab, avelumab, bms-936559, or atezolizumab.

In another aspect, provided herein is a method of treating a human patient having or suspected of having a pathogen or cancer, comprising: (1) administering to the human patient a stimulatory immune checkpoint activator; and (2) administering to the human patient a population of human cells comprising antigen-specific T cells that are specific for an antigen of the pathogen or cancer and are derived from a T cell line restricted by a first HLA allele or HLA allele combination expressed by the diseased cells in the human patient; wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

In specific embodiments, the stimulatory immune checkpoint activator is an activator of CD27, CD28, CD40, CD122, CD137, OX40, Glucocorticoid-Induced TNFR-Related Protein Ligand (GITR), or Inducible T-Cell Costimulator (ICOS).

In specific embodiments, the first HLA allele or HLA allele combination is classified as a subdominant HLA allele or HLA allele combination based on being associated with an indication of relative activity based on recognition of the antigen that is lower than the relative activity associated with a second HLA allele or HLA allele combination expressed by the diseased cells according to a representation, which representation (i) identifies a plurality of HLA alleles and optionally HLA allele combinations, and (ii) discloses indications of relative activities of T cell lines, each recognizing at least one epitope of the antigen, and restricted by different ones of the HLA alleles or HLA allele combinations in the plurality; wherein in the representation each identified HLA allele or HLA allele combination is associated with the respective indication of relative activity of the T cell line restricted by the HLA allele or HLA allele combination, the relative activities being relative measures of known activity of the T cell lines based on recognition of the antigen. In certain embodiments, a T cell line restricted by the second HLA allele or HLA allele combination is available and is suitable for therapeutic administration to the human patient.

In another specific aspect, provided herein is a method of selecting a T cell line for therapeutic administration, in combination with administration of an inhibitory immune checkpoint inhibitor or a stimulatory immune checkpoint activator, to a human patient having or suspected of having a pathogen or cancer, comprising: selecting a T cell line that recognizes at least one epitope of an antigen of the pathogen or cancer and is restricted by a first HLA allele or HLA allele combination expressed by the diseased cells in the human patient, wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination.

In specific embodiments, the first HLA allele or HLA allele combination is classified as a subdominant HLA allele or HLA allele combination based on being associated with an indication of relative activity based on recognition of the antigen that is lower than the relative activity associated with a second HLA allele or HLA allele combination expressed by the diseased cells according to a representation, which representation (i) identifies a plurality of HLA alleles and optionally HLA allele combinations, and (ii) discloses indications of relative activities of T cell lines, each recognizing at least one epitope of the antigen, and restricted by different ones of the HLA alleles or HLA allele combinations in the plurality; wherein in the Representation of Activity each identified HLA allele or HLA allele combination is associated with the respective indication of relative activity of the T cell line restricted by the HLA allele or HLA allele combination, the relative activities being relative measures of known activity of the T cell lines based on recognition of the antigen. In certain embodiments, a T cell line restricted by the second HLA allele or HLA allele combination is available and is suitable for therapeutic administration to the human patient.

In another aspect, provided herein is a method of treating a human patient having or suspected of having a pathogen or cancer, comprising: (a) selecting a T cell line for therapeutic administration to the human patient according to a method of selecting a T cell line as described herein; (b) administering to the human patient a population of human cells comprising antigen-specific T cells that are specific for the antigen and are derived from the selected T cell line; and (c) administering to the human patient an inhibitory immune checkpoint inhibitor.

In specific embodiments, the inhibitory immune checkpoint inhibitor is an inhibitor of Programmed Cell Death 1 (PD1), Programmed Death Ligand 1 (PD-L1), Programmed Death Ligand 1 (PD-L2), Cytotoxic T-Lymphocyte Associated Protein 4 (CTLA4), Lymphocyte Activating 3 (LAG3), T-Cell Immunoglobulin And Mucin Domain-Containing Protein 3 (TIM3), V-Domain Ig Suppressor Of T Cell Activation (VISTA), Adenosine A2a Receptor (A2aR), B7 Homolog 3 (B7-H3), B7 Homolog 4 (B7-H4), B and T lymphocyte associated (BTLA), Indoleamine 2,3-Dioxygenase (IDO), Tryptophan 2,3-Dioxygenase (TDO), or Killer-Cell Immunoglobulin-Like Receptor (KIR).

In specific embodiments, the inhibitory immune checkpoint inhibitor is an antibody that binds to and inhibits the activity of the inhibitory immune checkpoint. In a specific embodiment, the antibody is a monoclonal antibody.

In specific embodiments, the inhibitory immune checkpoint inhibitor is an inhibitor of PD1. In a specific embodiment, the inhibitory immune checkpoint inhibitor is a monoclonal antibody that binds to and inhibits the activity of PD1. In a particular embodiment, the monoclonal antibody is nivolumab, pidilizumab, MEDI0680, or pembrolizumab.

In specific embodiments, the inhibitory immune checkpoint inhibitor is an inhibitor of PD-L1. In a specific embodiment, the inhibitory immune checkpoint inhibitor is a monoclonal antibody that binds to and inhibits the activity of PD-L1. In a particular embodiment, the monoclonal antibody is mpd13280A, durvalumab, avelumab, bms-936559, or atezolizumab.

In another aspect, provided herein is a method of treating a human patient having or suspected of having a pathogen or cancer, comprising: (a) selecting a T cell line for therapeutic administration to the human patient according to a method of selecting a T cell line as described herein; (b) administering to the human patient a population of human cells comprising antigen-specific T cells that are specific for the antigen and are derived from the selected T cell line; and (c) administering to the human patient a stimulatory immune checkpoint activator.

In specific embodiments, the stimulatory immune checkpoint activator is an activator of CD27, CD28, CD40, CD122, CD137, OX40, Glucocorticoid-Induced TNFR-Related Protein Ligand (GITR), or Inducible T-Cell Costimulator (ICOS).

In another aspect, provided herein is a method of selecting a T cell donor from whom to derive a T cell line for therapeutic administration, in combination with administration of an inhibitory immune checkpoint inhibitor or a stimulatory immune checkpoint activator, to a human patient having or suspected of having a pathogen or cancer, comprising: selecting a T cell donor, using a first representation that (i) identifies a first plurality of HLA alleles and optionally HLA allele combinations, and (ii) discloses indications of relative frequencies of generation of T cell lines, each recognizing at least one epitope of an antigen of the pathogen or the cancer, and restricted by different ones of said HLA alleles or HLA allele combinations in the first plurality; wherein in the first representation each identified HLA allele or HLA allele combination is associated with the respective indication of relative frequency of generation of said T cell lines restricted by the respective HLA allele or HLA allele combination; wherein: (A) the T cell donor selected has a first HLA allele or HLA allele combination in common with the diseased cells in the human patient that is associated in the first representation with an indication of the highest frequency of generation; and (B) the first HLA allele or HLA allele combination of the selected T cell donor is a subdominant HLA allele or HLA allele combination among HLA alleles and HLA allele combinations expressed by the diseased cells with respect to activity of T cells restricted by the respective HLA allele or HLA allele combination based on recognition of the antigen.

In specific embodiments, the first HLA allele or HLA allele combination is classified as a subdominant HLA allele or HLA allele combination based on being associated with an indication of relative activity that is lower than the relative activity associated with a second HLA allele or HLA allele combination of the diseased cells according to a second representation, which second representation (I) identifies a second plurality of HLA alleles and optionally HLA allele combinations, and (II) discloses indications of relative activities of T cell lines, each recognizing at least one epitope of an antigen, and restricted by different ones of the HLA alleles or HLA allele combinations in the second plurality; wherein in the second representation each identified HLA allele or HLA allele combination is associated with the respective indication of relative activity of the T cell line restricted by the HLA allele or HLA allele combination, the relative activities being relative measures of known activity of the T cell lines based on recognition of the antigen.

In specific embodiments, the T cell donor is allogeneic to the human patient. In a specific embodiment, the human patient has been the recipient of a transplant from a transplant donor, and the T cell donor is a third party donor that is different from the transplant donor.

In another aspect, provided herein is a method of obtaining a T cell line for therapeutic administration, in combination with administration of an inhibitory immune checkpoint inhibitor or a stimulatory immune checkpoint activator, to a human patient having or suspected of having a pathogen or cancer, comprising: (a) selecting a T cell donor according to a method of selecting a T cell donor described herein; and (b) generating a T cell line from the selected T cell donor, which T cell line is restricted by the first HLA allele or HLA allele combination and recognizes at least one epitope of the antigen.

In specific embodiments of the methods of treating and methods of selecting a T cell line described herein, the T cell line is derived from a human donor that is allogeneic to the human patient. In a specific embodiment of the methods of treating and methods of selecting a T cell line described herein, the human patient has been the recipient of a transplant from a transplant donor, and the human donor is a third party donor that is different from the transplant donor.

In specific embodiments of the methods of treating and methods of selecting a T cell line described herein, the activity of T cells is in vitro cytotoxic activity of the T cells against cells expressing the antigen. In specific embodiments of the methods of treating and methods of selecting a T cell line described herein, the activity of T cells is in vivo clinical efficacies of the T cells in treatment of human patients having the pathogen or cancer.

In specific embodiments of the methods of treating and methods of selecting a T cell line described herein, a method of treating or a method of selecting a T cell line described herein further comprises a step of generating the T cell line restricted by the first HLA allele or HLA allele combination. In a specific embodiment, the step of generating the T cell line restricted by the first HLA allele or HLA allele combination comprises ex vivo sensitizing T cells to the antigen.

In specific embodiments of the methods of treating and methods of selecting a T cell line described herein, the T cell line lacks substantial cytotoxicity in vitro toward antigen presenting cells that are not loaded with or genetically engineered to express one or more peptides or proteins derived from the antigen of the pathogen or cancer.

In specific embodiments of the methods of treating, methods of selecting a T cell line, methods of selecting a donor, and methods of obtaining a T cell line described herein, the method further comprises a step of ascertaining the HLA assignment of the diseased cells in the human patient. In a specific embodiment, the step of ascertaining comprises typing at least 4 HLA loci.

In specific embodiments of the methods of treating, methods of selecting a T cell line, methods of selecting a donor, and methods of obtaining a T cell line described herein, the antigen is an antigen of a pathogen. In specific embodiments, the pathogen is a virus, bacterium, fungus, helminth or protist.

In a specific embodiment, the pathogen is a virus. In a particular embodiment, the virus is cytomegalovirus (CMV). In another particular embodiment, the virus is Epstein-Barr virus (EBV). In another particular embodiment, the virus is BK virus (BKV), John Cunningham virus (JCV), human herpesvirus, human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

In specific embodiments of the methods of treating, methods of selecting a T cell line, methods of selecting a donor, and methods of obtaining a T cell line described herein, the antigen is an antigen of a cancer. In specific embodiments, the antigen is Wilms Tumor 1 (WT1).

In another aspect, provided herein is a pharmaceutical composition comprising: (1) an inhibitory immune checkpoint inhibitor; and (2) a population of human cells comprising antigen-specific T cells that are specific for an antigen of the pathogen or cancer and are derived from a T cell line restricted by a first HLA allele or HLA allele combination; wherein the first HLA allele or HLA allele combination is a subdominant HLA allele or HLA allele combination with respect to activity of T cells restricted by the respective HLA allele or HLA allele combination based on recognition of the antigen.

In specific embodiments, the inhibitory immune checkpoint inhibitor is an inhibitor of Programmed Cell Death 1 (PD1), Programmed Death Ligand 1 (PD-L1), Programmed Death Ligand 1 (PD-L2), Cytotoxic T-Lymphocyte Associated Protein 4 (CTLA4), Lymphocyte Activating 3 (LAG3), T-Cell Immunoglobulin And Mucin Domain-Containing Protein 3 (TIM3), V-Domain Ig Suppressor Of T Cell Activation (VISTA), Adenosine A2a Receptor (A2aR), B7 Homolog 3 (B7-H3), B7 Homolog 4 (B7-H4), B and T lymphocyte associated (BTLA), Indoleamine 2,3-Dioxygenase (IDO), Tryptophan 2,3-Dioxygenase (TDO), or Killer-Cell Immunoglobulin-Like Receptor (KIR).

In another aspect, provided herein is a pharmaceutical composition comprising: (1) a stimulatory immune checkpoint activator; and (2) a population of human cells comprising antigen-specific T cells that are specific for an antigen of the pathogen or cancer and are derived from a T cell line restricted by a first HLA allele or HLA allele combination; wherein the first HLA allele or HLA allele combination is a subdominant HLA allele or HLA allele combination with respect to activity of T cells restricted by the respective HLA allele or HLA allele combination based on recognition of the antigen.

In specific embodiments, the stimulatory immune checkpoint activator is an activator of CD27, CD28, CD40, CD122, CD137, OX40, Glucocorticoid-Induced TNFR-Related Protein Ligand (GITR), or Inducible T-Cell Costimulator (ICOS).

In specific embodiments of the pharmaceutical compositions described herein, the first HLA allele or HLA allele combination is classified as a subdominant HLA allele or HLA allele combination based on being associated with an indication of lower activity based on recognition of the antigen that is lower than the relative activity associated with a second HLA allele or HLA allele combination according to a representation, which representation (i) identifies a plurality of HLA alleles and optionally HLA allele combinations, and (ii) discloses indications of relative activities of T cell lines, each recognizing at least one epitope of an antigen, and restricted by different ones of the HLA alleles or HLA allele combinations in the plurality; wherein in the representation each identified HLA allele or HLA allele combination is associated with the respective indication of relative activity of the T cell line restricted by the HLA allele or HLA allele combination, the relative activities being relative measures of known activity of the T cell lines based on recognition of the antigen.

In specific embodiments of the pharmaceutical compositions described herein, the antigen is an antigen of a pathogen. In specific embodiments, the pathogen is a virus, bacterium, fungus, helminth or protist.

In a specific embodiment of the pharmaceutical compositions described herein, the pathogen is a virus. In a particular embodiment, the virus is cytomegalovirus (CMV). In another particular embodiment, the virus is Epstein-Barr virus (EBV). In another particular embodiment, the virus is BK virus (BKV), John Cunningham virus (JCV), human herpesvirus, human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

In specific embodiments of the pharmaceutical compositions described herein, the antigen is an antigen of a cancer. In specific embodiments, the antigen is Wilms Tumor 1 (WT1).

In specific embodiments of the pharmaceutical compositions described herein, the T cell line lacks substantial cytotoxicity in vitro toward antigen presenting cells that are not loaded with or genetically engineered to express one or more peptides or proteins derived from the antigen of the pathogen or cancer.

### 4. BRIEF DESCRIPTION OF FIGURES

FIG. 1 shows the tumor weights under different treatment conditions.

FIG. 2 is a representation that depicts the percentage of interferon-γ-secreting CD3+ cells for each T cell line in a bank of 119 CMV-specific CTL lines that are restricted by HLA alleles or HLA allele combinations presenting immunodominant epitopes, clustered by their respective HLA alleles or HLA allele combinations, as described in Example, Section 6.2.2.3.

### 5. DETAILED DESCRIPTION

The present invention relates to methods of treating a human patient using a combination therapy comprising administering antigen-specific T cells derived from a T cell line that is restricted by a subdominant HLA allele (or HLA allele combination) and administering an immune checkpoint modulator (i.e., an inhibitory immune checkpoint inhibitor or a stimulatory immune checkpoint activator). The present invention further relates to methods of selecting such a T cell line, methods of selecting a T cell donor from whom to derive such a T cell line, and pharmaceutical compositions comprising such antigen-specific T cells and an immune checkpoint modulator. According to the invention, the immune checkpoint modulator augments the efficacy of antigen-specific T cells restricted by the subdominant HLA allele (or subdominant HLA allele combination).

An HLA allele (or HLA allele combination) is termed “subdominant” with respect to a particular human patient or human patient population having a pathogen or cancer expressing a particular antigen (an antigen of interest) when T cells restricted by that HLA allele (or HLA allele combination) do not have the highest T cell activity based on recognition of the antigen, among all of the HLA alleles (or HLA allele combinations) expressed by the diseased cells in the human patient or the human patient population. The T cell activity preferably is in vivo clinical efficacy obtained by administration of T cells restricted by the particular HLA allele (or HLA allele combination); in other embodiments, the T cell activity can be in vitro activity (for example, cytotoxic activity or IFN-γ production activity) of the T cells restricted by the particular HLA allele (or HLA allele combination).

5.1. Methods of Treatment

In one aspect, provided herein is a method of treating a human patient having or suspected of having a pathogen or cancer, comprising: (1) administering to the human patient an inhibitory immune checkpoint inhibitor; and (2) administering to the human patient a population of human cells comprising antigen-specific T cells that are specific for an antigen of the pathogen or cancer and are derived from a T cell line restricted by a first HLA allele or HLA allele combination expressed by the diseased cells in the human patient; wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In various embodiments, the human patient is in need of the treating. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

In another aspect, provided herein is a method of treating a human patient having or suspected of having a pathogen or cancer, comprising: (1) administering to the human patient a stimulatory immune checkpoint activator; and (2) administering to the human patient a population of human cells comprising antigen-specific T cells that are specific for an antigen of the pathogen or cancer and are derived from a T cell line restricted by a first HLA allele or HLA allele combination expressed by the diseased cells in the human patient; wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In various embodiments, the human patient is in need of the treating. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

When the human patient has a cancer, the diseased cells are the cancerous cells. When the human patient has a pathogen, the diseased cells are the cells infected by the pathogen.

Activity of T cells restricted by the respective HLA allele or HLA allele combination based on recognition of the antigen can be measured by using T cell lines generated in the same way, for example, generated by a method described in Section 5.4.

A T cell line may contain cells other than T cells. However, preferably, a T cell line is enriched for T cells (e.g., comprises more than 70%, more than 75%, more than 80%, more than 85%, more than 90%, more than 95%, or more than 99% T cells). A T cell line can be a collection of primary cells or a collection of cultured cells. Cells in the T cell line can be developed from a single cell or from multiple cells. In various embodiments, a T cell line is derived from a single human donor.

In specific embodiments, the first HLA allele or HLA allele combination is classified as a subdominant HLA allele or HLA allele combination based on being associated with an indication of relative activity based on recognition of the antigen that is lower than the relative activity associated with a second HLA allele or HLA allele combination expressed by the diseased cells according to a representation (hereinafter “Representation of Activity”), which representation (i) identifies a plurality of HLA alleles and optionally HLA allele combinations, and (ii) discloses indications of relative activities of T cell lines, each recognizing at least one epitope of the antigen, and restricted by different ones of the HLA alleles or HLA allele combinations in the plurality; wherein in the representation each identified HLA allele or HLA allele combination is associated with the respective indication of relative activity of the T cell line restricted by the HLA allele or HLA allele combination, the relative activities being relative measures of known activity of the T cell lines based on recognition of the antigen. In a specific embodiment, the first HLA allele or HLA allele combination is associated with an indication of no activity based on recognition of the antigen according to the Representation of Activity. An indication of no activity based on recognition of the antigen can be no detectable activity as measured in the generation of the Representation of Activity, or can be an indication of activity that is detectable but is deemed by one of ordinary skill in the art to be indicative of no clinical efficacy. In certain embodiments, a T cell line restricted by the second HLA allele or HLA allele combination is available and is suitable for therapeutic administration to the human patient.

In specific embodiments, the epitope of the antigen that is bound to the HLA protein encoded by the first HLA allele has the highest binding affinity for the first HLA allele among all epitopes of the antigen. In a specific embodiment, the epitope has the highest binding affinity for the first HLA allele among all epitopes of all antigens that can be presented by the first HLA allele.

As discussed above, in one embodiment, an HLA allele or HLA allele combination is determined to be subdominant based on comparisons among HLA alleles and HLA allele combinations that are suitable for therapeutic administration to the human patient. T cells are suitable for therapeutic administration to the human patient when a T cell line restricted by the respective HLA allele or HLA allele combination is available and is suitable for therapeutic administration. For example, if a T cell line is observed to have no or too few viable cells in the cell line sample (or after the cell line sample is thawed after freezing), the T cell line is unsuitable for therapeutic administration. As but another example, if the relative activities in the Representation of Activity are based upon in vitro or ex vivo assays of activity and it is known that the relative in vivo activity of a T cell line restricted by a particular HLA allele or HLA allele combination does not correlate with the relative in vitro or ex vivo assay used for generating the Representation of Activity, such that the highest relative activity in the Representation of Activity is not the highest relative in vivo activity, the particular HLA allele or HLA allele combination (by which such T cell line is restricted) can be deemed unsuitable for therapeutic administration. For example, it has been observed that the in vivo activity against CMV infection in human patients for T cell lines restricted by HLA-B35 is clinically ineffective (therefore negligible relative in vivo activity), although the percentage of interferon-γ-secreting CD3+ T cells derived from T cell lines restricted by HLA-B35 indicates a much higher relative activity; thus, in the context of treating CMV infections, in a specific embodiment, a T cell line restricted by HLA-B35 is deemed unsuitable for therapeutic administration. In a related specific embodiment, a T cell line restricted by a particular HLA allele or HLA allele combination is deemed unsuitable for therapeutic administration if T cell line(s) restricted by the particular HLA allele or HLA allele combination are known to be clinically ineffective in treatment of human patients having the pathogen or cancer. In alternative specific embodiments, however, a T cell line restricted by a particular HLA allele or HLA allele combination is not deemed as unsuitable if T cell line(s) restricted by the particular HLA allele or HLA allele combination are known to be clinically ineffective in treatment of human patients having the pathogen or cancer (thus, in the context of treating CMV infections, a T cell line restricted by HLA-B35 is not deemed unsuitable for therapeutic administration in these other specific embodiments), since the combination therapy of the invention may augment efficacy of such T cell line.

In another aspect, provided herein is a method of treating a human patient having or suspected of having a pathogen or cancer, comprising: (a) selecting a T cell line for therapeutic administration to the human patient according to a method described in Section 5.2; (b) administering to the human patient a population of human cells comprising antigen-specific T cells that are specific for the antigen and are derived from the selected T cell line; and (c) administering to the human patient an inhibitory immune checkpoint inhibitor.

In another aspect, provided herein is a method of treating a human patient having or suspected of having a pathogen or cancer, comprising: (a) selecting a T cell line for therapeutic administration to the human patient according to a method described in Section 5.2; (b) administering to the human patient a population of human cells comprising antigen-specific T cells that are specific for the antigen and are derived from the selected T cell line; and (c) administering to the human patient a stimulatory immune checkpoint activator.

In some embodiments of the aspects and embodiments described herein, the activity of T cells (and the relative activities of T cell lines, when a Representative of Activity is used) is in vitro antigen reactivity (for example, cytotoxic activity) of the T cells (or T cell lines, as the case may be) against the antigen (for example, against cells expressing the antigen). The in vitro antigen reactivity (for example, cytotoxic activity) of the T cells (or T cell lines, as the case may be) can be measured as described in Section 5.4.1. In other embodiments of the aspects and embodiments described herein, the activity of T cells (and the relative activities of T cell lines, when a Representative of Activity is used) is in vivo clinical efficacies of the T cells (or T cell lines, as the case may be) in treatment of human patients having the pathogen or cancer.

In specific embodiments when a Representation of Activity is used, the method of treating further comprises, prior to the administering steps, a step of generating the Representation of Activity using a method described in Section 5.7.

5.1.1. Administration and Dosage

The route of administration of the population of human cells comprising antigen-specific T cells, the route of administration of the inhibitory immune checkpoint inhibitor or the stimulatory immune checkpoint activator, and their respective amount to be administered to the human patient can be determined based on the nature of the disease, condition of the human patient and the knowledge of the physician. Generally, the administration of the population of human cells is intravenous. In certain embodiments, the method of treating comprises infusing to the human patient the population of human cells comprising antigen-specific T cells. In specific embodiments, the infusing is by bolus intravenous infusion. The inhibitory immune checkpoint inhibitor or the stimulatory immune checkpoint activator may be administered to human patients by a variety of routes, including, but are not limited to, parenteral, intranasal, intratracheal, oral, intradermal, topical, intramuscular, intraperitoneal, transdermal, intravenous, intratumoral, conjunctival, subcutaneous, and pulmonary routes. In specific embodiments, the administration of the inhibitory immune checkpoint inhibitor or the stimulatory immune checkpoint activator is intravenous.

In specific embodiments, the population of human cells comprising antigen-specific T cells is administered at a dose that is lower than the dose effective for treating the human patient if administered alone (for example, a dose used in standard-of-care therapy). In specific embodiments, the inhibitory immune checkpoint inhibitor or the stimulatory immune checkpoint activator is administered at a dose that is lower than the dose effective for treating the human patient if administered alone (for example, a dose used in standard-of-care therapy).

In some embodiments, the population of human cells comprising antigen-specific T cells and the inhibitory immune checkpoint inhibitor or the stimulatory immune checkpoint activator are administered concurrently, when the human patient is administered one of the two therapies when still being subject to the effect of the other therapy, for example, at about the same time, the same day, or same week, or same treatment cycle, or on similar dosing schedules, or on different but overlapping dosing schedules. In a specific embodiment, the population of human cells comprising antigen-specific T cells and the inhibitory immune checkpoint inhibitor or the stimulatory immune checkpoint activator are administered simultaneously. In other embodiments, the population of human cells comprising antigen-specific T cells and the inhibitory immune checkpoint inhibitor or the stimulatory immune checkpoint activator are administered separately, when the human patient is administered one of the two therapies when no longer being subject to the effect of the other therapy. In certain embodiments, the population of human cells comprising antigen-specific T cells and the inhibitory immune checkpoint inhibitor or the stimulatory immune checkpoint activator are administered sequentially.

In some embodiments, the population of human cells comprising antigen-specific T cells and the inhibitory immune checkpoint inhibitor or the stimulatory immune checkpoint activator are administered via a single composition. In other embodiments, the population of human cells comprising antigen-specific T cells and the inhibitory immune checkpoint inhibitor or the stimulatory immune checkpoint activator are administered via separate compositions.

In some embodiments, the method of treating comprises administering to the human patient the population of human cells comprising antigen-specific T cells, at a dose that is less than or equal to about 1×10⁵ cells of the population of human cells comprising antigen-specific T cells per kg of the human patient. In a specific embodiment, the method of treating comprises administering to the human patient the population of human cells comprising antigen-specific T cells, at a dose that is less than or equal to about 5×10⁴ cells of the population of human cells comprising antigen-specific T cells per kg of the human patient. In another specific embodiment, the method of treating comprises administering to the human patient the population of human cells comprising antigen-specific T cells, at a dose that is less than or equal to about 1×10⁴ cells of the population of human cells comprising antigen-specific T cells per kg of the human patient. In another specific embodiment, the method of treating comprises administering to the human patient the population of human cells comprising antigen-specific T cells, at a dose that is less than or equal to about 5×10³ cells of the population of human cells comprising antigen-specific T cells per kg of the human patient. In another specific embodiment, the method of treating comprises administering to the human patient the population of human cells comprising antigen-specific T cells, at a dose that is less than or equal to about 1×10³ cells of the population of human cells comprising antigen-specific T cells per kg of the human patient. In another specific embodiment, the method of treating comprises administering to the human patient the population of human cells comprising antigen-specific T cells, at a dose of about 1×10³ to 5×10³ cells of the population of human cells comprising antigen-specific T cells per kg of the human patient. In another specific embodiment, the method of treating comprises administering to the human patient the population of human cells comprising antigen-specific T cells, at a dose of about 5×10³ to 1×10⁴ cells of the population of human cells comprising antigen-specific T cells per kg of the human patient. In another specific embodiment, the method of treating comprises administering to the human patient the population of human cells comprising antigen-specific T cells, at a dose of about 1×10⁴ to 5×10⁴ cells of the population of human cells comprising antigen-specific T cells per kg of the human patient. In another specific embodiment, the method of treating comprises administering to the human patient the population of human cells comprising antigen-specific T cells, at a dose of about 5×10⁴ to 1×10⁵ cells of the population of human cells comprising antigen-specific T cells per kg of the human patient.

In other embodiments, the method of treating comprises administering to the human patient the population of human cells comprising antigen-specific T cells, at a dose that is at least 1×10⁵ cells of the population of human cells comprising antigen-specific T cells per kg of the human patient. In a specific embodiment, the method of treating comprises administering to the human patient the population of human cells comprising antigen-specific T cells, at a dose that is about 5×10⁵ cells of the population of human cells comprising antigen-specific T cells per kg of the human patient. In another specific embodiment, the method of treating comprises administering to the human patient the population of human cells comprising antigen-specific T cells, at a dose that is about 1×10⁶ cells of the population of human cells comprising antigen-specific T cells per kg of the human patient. In another specific embodiment, the method of treating comprises administering to the human patient the population of human cells comprising antigen-specific T cells, at a dose that is about 2×10⁶ cells of the population of human cells comprising antigen-specific T cells per kg of the human patient. In another specific embodiment, the method of treating comprises administering to the human patient the population of human cells comprising antigen-specific T cells, at a dose that is about 3×10⁶ cells of the population of human cells comprising antigen-specific T cells per kg of the human patient. In another specific embodiment, the method of treating comprises administering to the human patient the population of human cells comprising antigen-specific T cells, at a dose that is about 4×10⁶ cells of the population of human cells comprising antigen-specific T cells per kg of the human patient. In another specific embodiment, the method of treating comprises administering to the human patient the population of human cells comprising antigen-specific T cells, at a dose that is about 5×10⁶ cells of the population of human cells comprising antigen-specific T cells per kg of the human patient. In another specific embodiment, the method of treating comprises administering to the human patient the population of human cells comprising antigen-specific T cells, at a dose that is about 6×10⁶ cells of the population of human cells comprising antigen-specific T cells per kg of the human patient. In another specific embodiment, the method of treating comprises administering to the human patient the population of human cells comprising antigen-specific T cells, at a dose that is about 1×10⁷ cells of the population of human cells comprising antigen-specific T cells per kg of the human patient. In another specific embodiment, the method of treating comprises administering to the human patient the population of human cells comprising antigen-specific T cells, at a dose that is about 1×10⁵ to 5×10⁵ cells of the population of human cells comprising antigen-specific T cells per kg of the human patient. In another specific embodiment, the method of treating comprises administering to the human patient the population of human cells comprising antigen-specific T cells, at a dose that is about 5×10⁵ to 1×10⁶ cells of the population of human cells comprising antigen-specific T cells per kg of the human patient. In another specific embodiment, the method of treating comprises administering to the human patient the population of human cells comprising antigen-specific T cells, at a dose that is about 1×10⁶ to 2×10⁶ cells of the population of human cells comprising antigen-specific T cells per kg of the human patient. In another specific embodiment, the method of treating comprises administering to the human patient the population of human cells comprising antigen-specific T cells, at a dose that is about 2×10⁶ to 5×10⁶ cells of the population of human cells comprising antigen-specific T cells per kg of the human patient. In another specific embodiment, the method of treating comprises administering to the human patient the population of human cells comprising antigen-specific T cells, at a dose that is about 5×10⁶ to 1×10⁷ cells of the population of human cells comprising antigen-specific T cells per kg of the human patient.

In certain embodiments, the method of treating comprises administering to the human patient the population of human cells comprising antigen-specific T cells at the dose described above weekly. In certain embodiments, the method of treating comprises administering to the human patient the population of human cells comprising antigen-specific T cells at the dose described above twice weekly. In certain embodiments, the method of treating comprises administering to the human patient the population of human cells comprising antigen-specific T cells at the dose described above biweekly. In certain embodiments, the method of treating comprises administering to the human patient the population of human cells comprising antigen-specific T cells at the dose described above every three weeks.

In certain embodiments, the method of treating comprises administering to the human patient at least 2 doses of the population of human cells comprising antigen-specific T cells. In specific embodiments, the method of treating comprises administering to the human patient 2, 3, 4, 5, or 6 doses of the population of human cells comprising antigen-specific T cells. In a specific embodiment, the method of treating comprises administering to the human patient 2 doses of the population of human cells comprising antigen-specific T cells. In another specific embodiment, the method of treating comprises administering to the human patient 3 doses of the population of human cells comprising antigen-specific T cells. In another specific embodiment, the method of treating comprises administering to the human patient 4 doses of the population of human cells comprising antigen-specific T cells.

In specific embodiments, the method of treating comprises administering to the human patient at least two cycles (e.g., 2, 3, 4, 5, or 6 cycles) of one dose per week of the population of human cells comprising antigen-specific T cells for at least two consecutive weeks (e.g., 2, 3, 4, 5, or 6 consecutive weeks), each cycle separated by a washout period during which no dose of the population of human cells comprising antigen-specific T cells is administered. In a specific embodiment, the at least two consecutive weeks are 2 consecutive weeks. In a preferred embodiment, the at least two consecutive weeks are 3 consecutive weeks. In another specific embodiment, the at least two consecutive weeks are 4 consecutive weeks. In another specific embodiment, the method of treating comprises administering to the human patient two, three, four, five, or six cycles of one dose per week of the population of human cells comprising antigen-specific T cells for three consecutive weeks, each cycle separated by a washout period during which no dose of the population of human cells comprising antigen-specific T cells is administered. In another specific embodiment, the method of treating comprises administering to the human patient a first cycle of one dose per week of the population of human cells comprising antigen-specific T cells for 3 consecutive weeks followed by a washout period during which no dose of the population of human cells comprising antigen-specific T cells is administered, followed by a second cycle of said one dose per week of the population of human cells comprising antigen-specific T cells for 3 consecutive weeks. In specific embodiments, the washout period is at least about 1 week (e.g., about 1-6 weeks). In specific embodiments, the washout period is about 1, 2, 3, or 4 weeks. In a specific embodiment, the washout period is about 2 weeks. In a preferred embodiment, the washout period is about 3 weeks. In another specific embodiment, the washout period is about 4 weeks. Preferably, an additional cycle is administered only when the previous cycle has not exhibited toxicity (for example, no grade 3-5 serious adverse events, graded according to NCI CTCAE 4.0).

In specific embodiments, the method of treating comprises administering to the human patient continuously the population of human cells comprising antigen-specific T cells at a dose described herein weekly (i.e., there is no week during which the population of human cells comprising antigen-specific T cells is not administered, and thus there is no washout period).

In certain embodiments, a first dosage regimen described herein is carried out for a first period of time, followed by a second and different dosage regimen described herein that is carried out for a second period of time, wherein the first period of time and the second period of time are optionally separated by a washout period. In specific embodiments, the washout period is at least about 1 week (e.g., about 1-6 weeks). In specific embodiments, the washout period is about 1, 2, 3, or 4 weeks. In a specific embodiment, the washout period is about 2 weeks. In a preferred embodiment, the washout period is about 3 weeks. In another specific embodiment, the washout period is about 4 weeks. Preferably, the second dosage regimen is carried out only when the first dosage regimen has not exhibited toxicity (for example, no grade 3-5 serious adverse events, graded according to NCI CTCAE 4.0).

The term “about” shall be construed so as to allow normal variation.

5.1.2. Serial Treatment with Different Cell Populations

In certain embodiments, the method of treating a human patient having a pathogen or cancer as described above further comprises, after administering to the human patient a first population of human cells comprising antigen-specific T cells as described in Section 5.1.1, administering to the human patient a second population of human cells comprising antigen-specific T cells, wherein the antigen-specific T cells in the second population of human cells comprising antigen-specific T cells are restricted by a different HLA allele (different from the HLA allele by which antigen-specific cells contained in the first population of human cells comprising antigen-specific T cells are restricted) shared with the diseased cells in the human patient. In a specific embodiment, the method of treating a human patient having a pathogen or cancer comprises administering a first cycle of one dose per week of the first population of human cells comprising antigen-specific T cells, for at least two consecutive weeks (e.g., 2, 3, 4, 5, or 6 consecutive weeks), optionally followed by a washout period during which no dose of any population of human cells comprising antigen-specific T cells is administered, and followed by a second cycle of one dose per week of the second population of human cells comprising antigen-specific T cells for at least two consecutive weeks (e.g., 2, 3, 4, 5, or 6 consecutive weeks). In specific embodiments, the washout period is at least about 1 week (e.g., about 1-6 weeks). In specific embodiments, the washout period is about 1, 2, 3, or 4 weeks. In a specific embodiment, the washout period is about 2 weeks. In a preferred embodiment, the washout period is about 3 weeks. In certain embodiments, the human patient has no response, an incomplete response, or a suboptimal response (i.e., the human patient may still have a substantial benefit from continuing treatment, but has reduced chances of optimal long-term outcomes) after administering the first population of human cells comprising antigen-specific T cells and prior to administering the second population of human cells comprising antigen-specific T cells.

The first and second populations of human cells comprising antigen-specific T cells can each be administered by any route and any dosage regimen as described in Section 5.1.1, supra.

In specific embodiments, two populations of human cells comprising antigen-specific T cells that are each restricted (i.e., antigen-specific T cells in the two populations of human cells are each restricted) by a different HLA allele shared with the diseased cells in the human patient are administered serially. In specific embodiments, three populations of human cells comprising antigen-specific T cells that are each restricted (i.e., antigen-specific T cells in the three populations of human cells are each restricted) by a different HLA allele shared with the diseased cells in the human patient are administered serially. In specific embodiments, four populations of human cells comprising antigen-specific T cells that are each restricted (i.e., antigen-specific T cells in the four populations of human cells are each restricted) by a different HLA allele shared with the diseased cells in the human patient are administered serially. In specific embodiments, more than four populations of human cells comprising antigen-specific T cells that are each restricted (i.e., antigen-specific T cells in the more than four populations of human cells are each restricted) by a different HLA allele shared with the diseased cells in the human patient are administered serially.

5.2. Selection of T Cell Line

In another specific aspect, provided herein is a method of selecting a T cell line for therapeutic administration, in combination with administration of an inhibitory immune checkpoint inhibitor or a stimulatory immune checkpoint activator, to a human patient having or suspected of having a pathogen or cancer, comprising: selecting a T cell line that recognizes at least one epitope of an antigen of the pathogen or cancer and is restricted by a first HLA allele or HLA allele combination expressed by the diseased cells in the human patient, wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In various embodiments, the human patient is need of the therapeutic administration. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination.

When the human patient has a cancer, the diseased cells are the cancerous cells. When the human patient has a pathogen, the diseased cells are the cells infected by the pathogen.

Activity of T cells restricted by the respective HLA allele or HLA allele combination based on recognition of the antigen can be measured by using T cell lines generated in the same way, for example, generated by a method described in Section 5.4.

A T cell line may contain cells other than T cells. However, preferably, a T cell line is enriched for T cells (e.g., comprises more than 70%, more than 75%, more than 80%, more than 85%, more than 90%, more than 95%, or more than 99% T cells). A T cell line can be a collection of primary cells or a collection of cultured cells. Cells in the T cell line can be developed from a single cell or from multiple cells. In various embodiments, a T cell line is derived from a single human donor.

In specific embodiments, the first HLA allele or HLA allele combination is classified as a subdominant HLA allele or HLA allele combination based on being associated with an indication of relative activity based on recognition of the antigen that is lower than the relative activity associated with a second HLA allele or HLA allele combination expressed by the diseased cells according to a Representation of Activity, which Representation of Activity (i) identifies a plurality of HLA alleles and optionally HLA allele combinations, and (ii) discloses indications of relative activities of T cell lines, each recognizing at least one epitope of the antigen, and restricted by different ones of the HLA alleles or HLA allele combinations in the plurality; wherein in the Representation of Activity each identified HLA allele or HLA allele combination is associated with the respective indication of relative activity of the T cell line restricted by the HLA allele or HLA allele combination, the relative activities being relative measures of known activity of the T cell lines based on recognition of the antigen. In a specific embodiment, the first HLA allele or HLA allele combination is associated with an indication of no activity based on recognition of the antigen according to the Representation of Activity. An indication of no activity based on recognition of the antigen can be no detectable activity as measured in the generation of the Representation of Activity, or can be an indication of activity that is detectable but is deemed by one of ordinary skill in the art to be indicative of no clinical efficacy. In certain embodiments, a T cell line restricted by the second HLA allele or HLA allele combination is available and is suitable for therapeutic administration to the human patient.

In specific embodiments, the epitope of the antigen that is bound to the HLA protein encoded by the first HLA allele has the highest binding affinity for the first HLA allele among all epitopes of the antigen. In a specific embodiment, the epitope has the highest binding affinity for the first HLA allele among all epitopes of all antigens that can be presented by the first HLA allele.

As discussed above, in one embodiment, an HLA allele or HLA allele combination is determined to be subdominant based on comparisons among HLA alleles and HLA allele combinations that are suitable for therapeutic administration to the human patient. T cells are suitable for therapeutic administration to the human patient when a T cell line restricted by the respective HLA allele or HLA allele combination is available and is suitable for therapeutic administration. For example, if a T cell line is observed to have no or too few viable cells in the cell line sample (or after the cell line sample is thawed after freezing), the T cell line is unsuitable for therapeutic administration. As but another example, if the relative activities in the Representation of Activity are based upon in vitro or ex vivo assays of activity and it is known that the relative in vivo activity of a T cell line restricted by a particular HLA allele or HLA allele combination does not correlate with the relative in vitro or ex vivo assay used for generating the Representation of Activity, such that the highest relative activity in the Representation of Activity is not the highest relative in vivo activity, the particular HLA allele or HLA allele combination (by which such T cell line is restricted) can be deemed unsuitable for therapeutic administration. For example, it has been observed that the in vivo activity against CMV infection in human patients for T cell lines restricted by HLA-B35 is clinically ineffective (therefore negligible relative in vivo activity), although the percentage of interferon-γ-secreting CD3+ T cells derived from T cell lines restricted by HLA-B35 indicates a much higher relative activity; thus, in the context of treating CMV infections, in a specific embodiment, a T cell line restricted by HLA-B35 is deemed unsuitable for therapeutic administration. In a related specific embodiment, a T cell line restricted by a particular HLA allele or HLA allele combination is deemed unsuitable for therapeutic administration if T cell line(s) restricted by the particular HLA allele or HLA allele combination are known to be clinically ineffective in treatment of human patients having the pathogen or cancer. In alternative specific embodiments, however, a T cell line restricted by a particular HLA allele or HLA allele combination is not deemed as unsuitable if T cell line(s) restricted by the particular HLA allele or HLA allele combination are known to be clinically ineffective in treatment of human patients having the pathogen or cancer (thus, in the context of treating CMV infections, a T cell line restricted by HLA-B35 is not deemed unsuitable for therapeutic administration in these other specific embodiments), since the combination therapy of the invention may augment efficacy of such T cell line.

In some embodiments of the aspects and embodiments described herein, the activity of T cells (and the relative activities of T cell lines, when a Representative of Activity is used) is in vitro antigen reactivity (for example, cytotoxic activity) of the T cells (or T cell lines, as the case may be) against the antigen (for example, against cells expressing the antigen). The in vitro antigen reactivity (for example, cytotoxic activity) of the T cells (or T cell lines, as the case may be) can be measured as described in Section 5.4.1. In other embodiments of the aspects and embodiments described herein, the activity of T cells (and the relative activities of T cell lines, when a Representative of Activity is used) is in vivo clinical efficacies of the T cells (or T cell lines, as the case may be) in treatment of human patients having the pathogen or cancer.

In specific embodiments when a Representation of Activity is used, the method of selecting a T cell line further comprises, prior to the selecting step, a step of generating the Representation of Activity using a method described in Section 5.7.

5.3. Selection of T Cell Donor

In another aspect, provided herein is a method of selecting a T cell donor from whom to derive a T cell line for therapeutic administration, in combination with administration of an inhibitory immune checkpoint inhibitor or a stimulatory immune checkpoint activator, to a human patient having or suspected of having a pathogen or cancer, comprising: selecting a T cell donor, using a first representation (hereinafter “Representation of Frequency”) that (i) identifies a first plurality of HLA alleles and optionally HLA allele combinations, and (ii) discloses indications of relative frequencies of generation of T cell lines, each recognizing at least one epitope of an antigen of the pathogen or the cancer, and restricted by different ones of said HLA alleles or HLA allele combinations in the first plurality; wherein in the first representation each identified HLA allele or HLA allele combination is associated with the respective indication of relative frequency of generation of said T cell lines restricted by the respective HLA allele or HLA allele combination; wherein: (A) the T cell donor selected has a first HLA allele or HLA allele combination in common with the diseased cells in the human patient that is associated in the first representation with an indication of the highest frequency of generation; and (B) the first HLA allele or HLA allele combination of the selected T cell donor is a subdominant HLA allele or HLA allele combination among HLA alleles and HLA allele combinations expressed by the diseased cells with respect to activity of T cells restricted by the respective HLA allele or HLA allele combination based on recognition of the antigen. In various embodiments, the human patient is need of the therapeutic administration.

When the human patient has a cancer, the diseased cells are the cancerous cells. When the human patient has a pathogen, the diseased cells are the cells infected by the pathogen.

Activity of T cells restricted by the respective HLA allele or HLA allele combination based on recognition of the antigen can be measured by using T cell lines generated in the same way, for example, generated by a method described in Section 5.4.

A T cell line may contain cells other than T cells. However, preferably, a T cell line is enriched for T cells (e.g., comprises more than 70%, more than 75%, more than 80%, more than 85%, more than 90%, more than 95%, or more than 99% T cells). A T cell line can be a collection of primary cells or a collection of cultured cells. Cells in the T cell line can be developed from a single cell or from multiple cells. In various embodiments, a T cell line is derived from a single human donor.

In specific embodiments, the first HLA allele or HLA allele combination is classified as a subdominant HLA allele or HLA allele combination based on being associated with an indication of relative activity that is lower than the relative activity associated with a second HLA allele or HLA allele combination of the diseased cells according to a Representation of Activity, which Representation of Activity (I) identifies a second plurality of HLA alleles and optionally HLA allele combinations, and (II) discloses indications of relative activities of T cell lines, each recognizing at least one epitope of an antigen, and restricted by different ones of the HLA alleles or HLA allele combinations in the second plurality; wherein in the Representation of Activity each identified HLA allele or HLA allele combination is associated with the respective indication of relative activity of the T cell line restricted by the HLA allele or HLA allele combination, the relative activities being relative measures of known activity of the T cell lines based on recognition of the antigen. In a specific embodiment, the first HLA allele or HLA allele combination is associated with an indication of no activity based on recognition of the antigen according to the Representation of Activity. An indication of no activity based on recognition of the antigen can be no detectable activity as measured in the generation of the Representation of Activity, or can be an indication of activity that is detectable but is deemed by one of ordinary skill in the art to be indicative of no clinical efficacy.

In specific embodiments, the epitope of the antigen that is bound to the HLA protein encoded by the first HLA allele has the highest binding affinity for the first HLA allele among all epitopes of the antigen. In a specific embodiment, the epitope has the highest binding affinity for the first HLA allele among all epitopes of all antigens that can be presented by the first HLA allele.

In specific embodiments, the T cell donor is allogeneic to the human patient. In a specific embodiment, the human patient has been the recipient of a transplant from a transplant donor, and the T cell donor is a third party donor that is different from the transplant donor. In another specific embodiment, the human patient has been the recipient of a transplant from a transplant donor, and the T cell donor is the transplant donor. In some embodiments, the transplant is a hematopoietic stem cell transplantation (HSCT), such as a peripheral blood stem cell transplantation, a bone marrow transplantation, or a cord blood transplantation. In other embodiments, the transplant is a solid organ transplant, such as a kidney transplant, a liver transplant, a heart transplant, an intestinal transplant, a pancreas transplant, a lung transplant, or a small bowel transplant, or a combination thereof (for example, a combination of heart transplant and lung transplant, or a combination of kidney transplant and pancreas transplant).

In some embodiments when a Representative of Activity is used, the relative activities of T cell lines are in vitro antigen reactivity (for example, cytotoxic activities) of the T cell lines based on recognition of the antigen. The in vitro antigen reactivity (for example, cytotoxic activities) of the T cell lines can be measured as described in Section 5.4.1. In other embodiments when a Representative of Activity is used, the relative activities of T cell lines are are in vivo clinical efficacies of the T cell lines in treatment of human patients having the pathogen or cancer.

In another aspect, provided herein is a method of obtaining a T cell line for therapeutic administration, in combination with administration of an inhibitory immune checkpoint inhibitor or a stimulatory immune checkpoint activator, to a human patient having or suspected of having a pathogen or cancer, comprising: (a) selecting a T cell donor according to a method of selecting a T cell donor described herein; and (b) generating a T cell line from the selected T cell donor, which T cell line is restricted by the first HLA allele or HLA allele combination and recognizes at least one epitope of the antigen.

In specific embodiments, the method of selecting a T cell donor further comprises, prior to the selecting step, a step of generating the Representation of Frequency using a method described in Section 5.8.

In specific embodiments when a Representation of Activity is used, the method of selecting a T cell donor further comprises, prior to the selecting step, a step of generating the Representation of Activity using a method described in Section 5.7.

5.4. Generation and Characteristics of Antigen-Specific T Cells

T cell lines used for therapeutic administration and/or for generation of a Representation of Activity or Representation of Frequency can be generated as described herein. In specific embodiments, a method of treating or a method of selecting a T cell line described herein further comprises a step of generating the T cell line restricted by the first HLA allele or HLA allele combination.

In some embodiments, the step of generating the T cell line restricted by the first HLA allele or HLA allele combination comprises ex vivo sensitizing T cells to the antigen. It is noted that the methods of performing the ex vivo sensitizing step described below can also be used to generate T cell lines used for generation of a Representation of Activity or Representation of Frequency as well.

The ex vivo sensitizing step can be performed by any method known in the art to stimulate T cells to be antigen-specific ex vivo, such as a method as described in Koehne et al., 2000, Blood 96:109-117; Trivedi et al., 2005, Blood 105:2793-2801; Haque et al., 2007, Blood 110:1123-1131; Hasan et al., 2009, J Immunol 183: 2837-2850; Feuchtinger et al., 2010, Blood 116:4360-4367; Doubrovina et al., 2012, Blood 120:1633-1646; Doubrovina et al., 2012, Blood 119:2644-2656; Leen et al., 2013, Blood 121:5113-5123; Papadopoulou et al., 2014, Sci Transl Med 6:242ra83; Sukdolak et al., 2013, Biol Blood Marrow Transplant 19:1480-1492; Koehne et al., 2015, Biol Blood Marrow Transplant 21: 1663-1678, or International Patent Application Publication No. WO 2016/073550.

In specific embodiments, the ex vivo sensitizing step comprises co-culturing T cells with one or more immunogenic peptides or proteins derived from the antigen (preferably also in the presence of antigen presenting cells). In specific embodiments, the ex vivo sensitizing step comprises co-culturing T cells with antigen presenting cells that present the antigen.

In a specific embodiment, the ex vivo sensitizing step comprises co-culturing isolated PBMCs with one or more immunogenic peptides or proteins derived from the antigen, and preferably also in the presence of antigen presenting cells. In another specific embodiment, the ex vivo sensitizing step comprises co-culturing isolated PBMCs with antigen presenting cells that present the antigen. PBMCs can be isolated from a blood sample by any method known in the art for isolating PBMCs from blood cells, such as by Ficoll-Hypaque centrifugation as described in Koehne et al., 2000, Blood 96:109-117; Trivedi et al., 2005, Blood 105:2793-2801.

In a specific embodiment, the ex vivo sensitizing step comprises co-culturing a cell population that is enriched for T cells with one or more immunogenic peptides or proteins derived from the antigen, and preferably also in the presence of antigen presenting cells. In another specific embodiment, the ex vivo sensitizing step comprises co-culturing a cell population that is enriched for T cells with antigen presenting cells that present the antigen. T cells can be enriched by any method known in the art to enrich T cells from blood cells (for example, from PBMCs). Non-limiting exemplary methods for enriching T cells can be found in Koehne et al., 2000, Blood 96:109-117; Trivedi et al., 2005, Blood 105:2793-2801; Hasan et al., 2009, J Immunol 183: 2837-2850; and Koehne et al., 2015, Biol Blood Marrow Transplant 21: 1663-1678. In specific embodiments, T cells are enriched from PBMCs by sorting the PBMCs using an anti-CD3 antibody. In specific embodiments, T cells are enriched from PBMCs by depleting of adherent monocytes and natural killer cells from the PBMCs.

The antigen presenting cells used in the ex vivo sensitizing step can be any antigen presenting cells suitable for presenting the antigen, such as dendritic cells, cytokine-activated monocytes, peripheral blood mononuclear cells (PBMCs), Epstein-Barr virus-transformed B-lymphoblastoid cell line cells (EBV-BLCL cells), or artificial antigen presenting cells (AAPCs). In a specific embodiment, the antigen presenting cells are dendritic cells. In another specific embodiment, the antigen presenting cells are PBMCs. In another specific embodiment, the antigen presenting cells are EBV-BLCL cells. In another specific embodiment, the antigen presenting cells are AAPCs. In certain embodiments, the antigen presenting cells are derived from the donor of the T cell line. The antigen presenting cells can be obtained by any method known in the art, such as the method(s) described in Koehne et al., 2000, Blood 96:109-117; Koehne et al., 2002, Blood 99:1730-1740; Trivedi et al., 2005, Blood 105:2793-2801; O'Reilly et al., 2007, Immunol Res 38:237-250; Hasan et al., 2009, J Immunol 183: 2837-2850; Barker et al., 2010, Blood 116:5045-5049; O'Reilly et al., 2011, Best Practice & Research Clinical Haematology 24:381-391; Doubrovina et al., 2012, Blood 120:1633-1646; Doubrovina et al., 2012, Blood 119:2644-2656; Koehne et al., 2015, Biol Blood Marrow Transplant 21: 1663-1678, or International Patent Application Publication No. WO 2016/073550.

In specific embodiments, the antigen presenting cells are loaded with one or more immunogenic peptides or proteins derived from the antigen. Non-limiting exemplary methods for loading antigen presenting cells with peptide(s) derived from antigen(s) can be found in Trivedi et al., 2005, Blood 105:2793-2801; Hasan et al., 2009, J Immunol 183: 2837-2850; and International Patent Application Publication No. WO 2016/073550. In specific embodiments, the antigen presenting cells are genetically engineered to recombinantly express one or more immunogenic peptides or proteins derived from the antigen. Any appropriate method known in the art for introducing nucleic acid vehicles into cells to express proteins, such as transduction or transformation, can be used to genetically engineer the antigen presenting calls to recombinantly express the one or more immunogenic peptides or proteins derived from the antigen.

In certain embodiments, the one or more immunogenic peptides or proteins are a pool of overlapping peptides derived from the antigen. In specific embodiments, the pool of overlapping peptides is a pool of overlapping pentadecapeptides. In certain embodiments, the one or more immunogenic peptides or proteins are one or more proteins derived from the antigen.

In specific embodiments, the T cells used for ex vivo sensitizing to generate the T cell line are derived from a CD34⁻ cell population from the T cell line donor, which CD34⁻ cell population is the product of a method comprising separating CD34⁺ cells from CD34⁻ cells in an apheresis collection (e.g., a leukapheresis collection) that comprises T cells from a human donor who is G-CSF mobilized, as described in U.S. Provisional Patent Application No. 62/307,240, filed Mar. 11, 2016, the disclosure of which is hereby incorporated by reference in its entirety.

In specific embodiments, the T cells used for ex vivo sensitizing to generate the T cell line are at least 50% stem cell-like memory T cells (T_{SCM} cells), at least 90% T_{SCM} cells, at least 95% T_{SCM} cells, at least 99% T_{SCM} cells, or 100% T_{SCM} cells, as described in U.S. Provisional Patent Application No. 62/399,311, filed Sep. 23, 2016, the disclosure of which is hereby incorporated by reference in its entirety.

In other embodiments, the step of generating the T cell line restricted by the first HLA allele or HLA allele combination comprises fluorescence activated cell sorting (FACS) for antigen-specific T cells from a population of blood cells from a human donor seropositive for the antigen, wherein the antigen-specific T cells are specific for the antigen. In a specific embodiment, the population of blood cells are peripheral blood mononuclear cells (PBMCs) isolated from a blood sample(s) obtained from the human donor. The fluorescence activated cell sorting can be performed by any method known in the art, which normally involves staining the population of blood cells with an antibody that recognizes the antigen before the sorting step.

In certain embodiments, the step of generating the T cell line further comprises cryopreserving a cell population comprising the ex vivo sensitized or FACS-sorted T cells for storage.

In certain embodiments, a method of treating described herein further comprises deriving the population of human cells comprising antigen-specific T cells from the T cell line. In a specific embodiment, the population of human cells comprising antigen-specific T cells is a faction of the T cell line. In another specific embodiment, the T cell line is cryopreserved, and the method of deriving comprises thawing the T cell line or a fraction thereof, and optionally expanding the thawed T cells from the T cell line in vitro to generate the population of human cells for therapeutic administration. In another specific embodiment, the T cell line is not cryopreserved, and the method of deriving comprises expanding T cells from the T cell line in vitro to generate the population of human cells for therapeutic administration.

In specific embodiments of the methods described herein, the T cell line is derived from a human donor that is allogeneic to the human patient. In a specific embodiment, the human patient has been the recipient of a transplant from a transplant donor, and the human donor is a third party donor that is different from the transplant donor. In another specific embodiment, the human patient has been the recipient of a transplant from a transplant donor, and the human donor is the transplant donor. In some embodiments, the transplant is a hematopoietic stem cell transplantation (HSCT), such as a peripheral blood stem cell transplantation, a bone marrow transplantation, or a cord blood transplantation. In other embodiments, the transplant is a solid organ transplant, such as a kidney transplant, a liver transplant, a heart transplant, an intestinal transplant, a pancreas transplant, a lung transplant, or a small bowel transplant, or a combination thereof (for example, a combination of heart transplant and lung transplant, or a combination of kidney transplant and pancreas transplant).

A T cell line described herein preferably (1) exhibits substantial antigen reactivity (for example, cytotoxicity) toward fully or partially HLA-matched (relative to the human donor of the T cell line) antigen presenting cells that are loaded with or genetically engineered to express one or more peptides or proteins derived from the antigen of the pathogen or cancer; (2) lacks substantial alloreactivity; and/or (3) is restricted by an HLA allele or HLA allele combination shared with the diseased cells in the human patient, and/or shares at least 2 HLA alleles (e.g., at least 2 out of 8 HLA alleles) with the diseased cells in the human patient. Thus, preferably, antigen reactivity (for example, cytotoxicity), alloreactivity, information as to which HLA allele(s) the T cell line is restricted, and/or the HLA assignment of the T cell line are measured by a method known in the art before administration to a human patient (for example, such a method as described in Koehne et al., 2000, Blood 96:109-117; Trivedi et al., 2005, Blood 105:2793-2801; Haque et al., 2007, Blood 110:1123-1131; Hasan et al., 2009, J Immunol 183: 2837-2850; Feuchtinger et al., 2010, Blood 116:4360-4367; Doubrovina et al., 2012, Blood 120:1633-1646; Doubrovina et al., 2012, Blood 119:2644-2656; Leen et al., 2013, Blood 121:5113-5123; Papadopoulou et al., 2014, Sci Transl Med 6:242ra83; Sukdolak et al., 2013, Biol Blood Marrow Transplant 19:1480-1492; Koehne et al., 2015, Biol Blood Marrow Transplant 21: 1663-1678; or International Patent Application Publication No. WO 2016/073550).

5.4.1. Cytotoxicity and Other Measures of Antigen Reactivity

The antigen reactivity (for example cytotoxicity) of a T cell line toward fully or partially HLA-matched (relative to the human donor of the T cell line) antigen presenting cells can be determined by any assay known in the art to measure T cell mediated antigen reactivity (for example, cytotoxicity), such as, but is not limited to, a method described in Nagorsen and Marincola, ed., 2005, Analyzing T Cell Responses: How to Analyze Cellular Immune Responses against Tumor Associated Antigens, Springer Netherlands. The assay can be performed using the T cell line directly, an aliquot thereof, or a precursor cell population that indicates the antigen reactivity (for example, cytotoxicity) of the T cell line. In a specific embodiment, the antigen reactivity (for example, cytotoxicity) is determined by a standard ⁵¹Cr release assay, an IFN-γ-production assay, a limiting dilution assay to measure CTL precursors (CTLps), a perforin release assay, a granzyme B release assay, or a CD107 mobilization assay, as described in Trivedi et al., 2005, Blood 105:2793-2801, Hasan et al., 2009, J Immunol 183: 2837-2850, Doubrovina et al., 2012, Blood 119:2644-2656; Koehne et al., 2000, Blood 96:109-117, Weren et al., J Immunol Methods, 289:17-26, Shafer-Weaver et al., 2003, J Transl Med 1:14, or Nagorsen and Marincola, ed., 2005, Analyzing T Cell Responses: How to Analyze Cellular Immune Responses against Tumor Associated Antigens, Springer Netherlands.

In certain embodiments, the T cell line exhibits substantial antigen reactivity (for example, cytotoxicity) in vitro toward (e.g., exhibits substantial lysis of) fully or partially HLA matched antigen presenting cells that are loaded with or genetically engineered to express one or more peptides or proteins derived from the antigen of the pathogen or cancer. Preferably, the fully or partially HLA-matched antigen presenting cells are fully HLA-matched antigen presenting cells (e.g., antigen presenting cells derived from the human donor). In specific embodiments, the T cell line exhibits lysis of greater than or equal to 20%, 25%, 30%, 35%, or 40% of the fully or partially HLA-matched antigen presenting cells that are loaded with or genetically engineered to express one or more peptides or proteins derived from the antigen of the pathogen or cancer. In a specific embodiment, the T cell line exhibits lysis of greater than or equal to 20% of the fully or partially HLA-matched antigen presenting cells that are loaded with or genetically engineered to express one or more peptides or proteins derived from the antigen of the pathogen or cancer.

Antigen presenting cells that can be used in the antigen reactivity (for example, cytotoxicity) assay include, but are not limited to, dendritic cells, phytohemagglutinin (PHA)-lymphoblasts, macrophages, B-cells that generate antibodies, EBV-BLCL cells, and artificial antigen presenting cells (AAPCs).

In specific embodiments, the fully or partially HLA-matched antigen presenting cells used in the antigen reactivity (for example, cytotoxicity) assay are loaded with a pool of peptides derived from the antigen of the pathogen or cancer. The pool of peptides, can be, for example, a pool of overlapping peptides (e.g., pentadecapeptides) spanning the sequence of the antigen of the pathogen or cancer.

5.4.2. Alloreactivity

Alloreactivity of a T cell line can be measured using an antigen reactivity (for example, cytotoxicity) assay known in the art to measure T cell mediated antigen reactivity (for example, cytotoxicity), such as, but is limited to, a standard ⁵¹Cr release assay, an IFN-γ-production assay, a limiting dilution assay to measure CTL precursors (CTLps), a perforin release assay, a granzyme B release assay, a CD107 mobilization assay, or any other antigen reactivity assay as described in Section 5.4.1, but with antigen presenting cells that are not loaded with or genetically engineered to express one or more peptides or proteins derived from the antigen of the pathogen or cancer, and/or HLA-mismatched (relative to the human donor of the population of human cells) antigen presenting cells. The assay can be performed using the T cell line directly, an aliquot thereof, or a precursor cell population that indicates the alloreactivity of the T cell line. A population of human cells comprising antigen-specific T cells derived from a T cell line that lacks substantial alloreactivity results generally in the absence of graft-versus-host disease (GvHD) when administered to a human patient.

In certain embodiments, the T cell line lacks substantial antigen reactivity (for example, cytotoxicity) in vitro toward antigen presenting cells that are not loaded with or genetically engineered to express one or more peptides or proteins derived from the antigen of the pathogen or cancer. In preferred embodiments, such antigen-presenting cells are fully or partially HLA-matched antigen presenting cells (relative to the human donor of the population of human blood cells) (e.g., antigen presenting cells derived from the human donor of the population of human blood cells). In specific embodiments, the T cell line lyses less than or equal to 15%, 10%, 5%, 2%, or 1% of antigen presenting cells that are not loaded with or genetically engineered to express one or more peptides or proteins derived from the antigen of the pathogen or cancer. In a specific embodiment, the T cell line lyses less than or equal to 10% of antigen presenting cells that are not loaded with or genetically engineered to express one or more peptides or proteins derived from the antigen of the pathogen or cancer. In another specific embodiment, the T cell line lyses less than or equal to 5% of antigen presenting cells that are not loaded with or genetically engineered to express one or more peptides or proteins derived from the antigen of the pathogen or cancer.

In certain embodiments, the T cell line lacks substantial antigen reactivity (for example, cytotoxicity) in vitro toward HLA-mismatched (relative to the human donor of the population of human blood cells) antigen presenting cells. In some embodiments, such antigen-presenting cells are loaded with or genetically engineered to express one or more peptides or proteins derived from the antigen of the pathogen or cancer. In other embodiments, such antigen-presenting cells are not loaded with or genetically engineered to express one or more peptides or proteins derived from the antigen of the pathogen or cancer. In specific embodiments, the T cell line lyses less than or equal to 15%, 10%, 5%, 2%, or 1% of HLA-mismatched (relative to the human donor of the population of human blood cells) antigen presenting cells. In a specific embodiment, the T cell line lyses less than or equal to 10% of HLA-mismatched (relative to the human donor of the population of human blood cells) antigen presenting cells. In another specific embodiment, the T cell line lyses less than or equal to 5% of HLA-mismatched (relative to the human donor of the population of human blood cells) antigen presenting cells.

In certain embodiments, the T cell line lacks substantial antigen reactivity (for example, cytotoxicity) in vitro toward antigen presenting cells that are not loaded with or genetically engineered to express one or more peptides or proteins derived from the antigen of the pathogen or cancer, as described above, and lacks substantial antigen reactivity (for example, cytotoxicity) in vitro toward HLA-mismatched antigen presenting cells as described above.

Antigen presenting cells that can be used in the alloreactivity assay include, but are not limited to, dendritic cells, phytohemagglutinin (PHA)-lymphoblasts, macrophages, B-cells that generate antibodies, EBV-BLCL cells, and artificial antigen presenting cells (AAPCs).

5.4.3. HLA Type

The HLA assignment (i.e., the HLA loci type) of a T cell line and/or the HLA assignment of the diseased cells in the human patient can be ascertained (i.e., typed) by any method known in the art for typing HLA alleles. The assignment of a T cell line can be performed using the T cell line directly, an aliquot thereof, or a precursor cell population that indicates the HLA assignment of the T cell line. Non-limiting exemplary methods for ascertaining the HLA assignment can be found in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Hurley, “DNA-based typing of HLA for transplantation.” in Leffell et al., eds., 1997, Handbook of Human Immunology, Boca Raton: CRC Press; Dunn, 2011, Int J Immunogenet 38:463-473; Erlich, 2012, Tissue Antigens, 80:1-11; Bontadini, 2012, Methods, 56:471-476; and Lange et al., 2014, BMC Genomics 15: 63. In specific embodiments, at least 4 HLA loci (preferably HLA-A, HLA-B, HLA-C, and HLA-DR) are typed. In a specific embodiment, 4 HLA loci (preferably HLA-A, HLA-B, HLA-C, and HLA-DR) are typed. In another specific embodiment, 6 HLA loci are typed. In another specific embodiment, 8 HLA loci are typed.

In general, high-resolution typing is preferable for HLA typing. The high-resolution typing can be performed by any method known in the art, for example, as described in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Flomenberg et al., Blood, 104:1923-1930; Kogler et al., 2005, Bone Marrow Transplant, 36:1033-1041; Lee et al., 2007, Blood 110:4576-4583; Erlich, 2012, Tissue Antigens, 80:1-11; Lank et al., 2012, BMC Genomics 13:378; or Gabriel et al., 2014, Tissue Antigens, 83:65-75.

In certain embodiments, the method of treating a human patient, the method of selecting a T cell line, or the method of treating a T cell donor, further comprises a step of ascertaining the HLA assignment of the diseased cells in the human patient. In specific embodiments, the HLA assignment of the diseased cells in the human patient is ascertained by typing the origin of the diseased cells (e.g., the human patient or a transplant donor for the human patient, as the case may be). The origin of the diseased cells can be determined by any method known in the art, for example, by analyzing variable tandem repeats (VTRs) (which is a method that uses unique DNA signature of small DNA sequences of different people to distinguish between the recipient and the donor of a transplant), or by looking for the presence or absence of chromosome Y if the donor and the recipient of a transplant are of different sexes (which is done by cytogenetics or by FISH (fluorescence in situ hybridization)).

The HLA allele by which the T cell line is restricted can be determined by any method known in the art, for example, as described in Trivedi et al., 2005, Blood 105:2793-2801; Barker et al., 2010, Blood 116:5045-5049; Hasan et al., 2009, J Immunol, 183:2837-2850; Doubrovina et al., 2012, Blood 120:1633-1646; Doubrovina et al., 2012, Blood 119:2644-2656; or International Patent Application Publication No. WO 2016/073550. The determination can be performed using the T cell line directly, an aliquot thereof, or a precursor cell population that indicates the HLA allele by which the T cell line is restricted.

In some embodiments, the T cell line is restricted by an HLA allele shared with the diseased cells in the human patient. In other embodiments, the T cell line share at least 2 HLA alleles (for example, at least 2 out of 8 HLA alleles, such as two HLA-A alleles, two HLA-B alleles, two HLA-C alleles, and two HLA-DR alleles) with the diseased cells in the human patient. In other embodiments, the T cell line is restricted by an HLA allele shared with diseased cells in the human patient, and share at least 2 HLA alleles (for example, at least 2 out of 8 HLA alleles, such as two HLA-A alleles, two HLA-B alleles, two HLA-C alleles, and two HLA-DR alleles) with the diseased cells in the human patient.

5.5. Pharmaceutical Compositions

In another aspect, provided herein is a pharmaceutical composition comprising: (1) an inhibitory immune checkpoint inhibitor; and (2) a population of human cells comprising antigen-specific T cells that are specific for an antigen of the pathogen or cancer and are derived from a T cell line restricted by a first HLA allele or HLA allele combination; wherein the first HLA allele or HLA allele combination is a subdominant HLA allele or HLA allele combination with respect to activity of T cells restricted by the respective HLA allele or HLA allele combination based on recognition of the antigen.

In another aspect, provided herein is a pharmaceutical composition comprising: (1) a stimulatory immune checkpoint activator; and (2) a population of human cells comprising antigen-specific T cells that are specific for an antigen of the pathogen or cancer and are derived from a T cell line restricted by a first HLA allele or HLA allele combination; wherein the first HLA allele or HLA allele combination is a subdominant HLA allele or HLA allele combination with respect to activity of T cells restricted by the respective HLA allele or HLA allele combination based on recognition of the antigen.

In specific embodiments of the pharmaceutical composition, the first HLA allele or HLA allele combination is classified as a subdominant HLA allele or HLA allele combination based on being associated with an indication of lower activity based on recognition of the antigen that is lower than the relative activity associated with a second HLA allele or HLA allele combination according to a Representation of Activity, which Representation of Activity (i) identifies a plurality of HLA alleles and optionally HLA allele combinations, and (ii) discloses indications of relative activities of T cell lines, each recognizing at least one epitope of an antigen, and restricted by different ones of the HLA alleles or HLA allele combinations in the plurality; wherein in the Representation of Activity each identified HLA allele or HLA allele combination is associated with the respective indication of relative activity of the T cell line restricted by the HLA allele or HLA allele combination, the relative activities being relative measures of known activity of the T cell lines based on recognition of the antigen. In a specific embodiment, the first HLA allele or HLA allele combination is associated with an indication of no activity based on recognition of the antigen according to the Representation of Activity. An indication of no activity based on recognition of the antigen can be no detectable activity as measured in the generation of the Representation of Activity, or can be an indication of activity that is detectable but is deemed by one of ordinary skill in the art to be indicative of no clinical efficacy.

In another aspect, provided herein is a pharmaceutical composition for therapeutic administration to a human patient having or suspected of having a pathogen or cancer, comprising: (1) an inhibitory immune checkpoint inhibitor; and (2) a population of human cells comprising antigen-specific T cells that are specific for an antigen of the pathogen or cancer and are derived from a T cell line restricted by a first HLA allele or HLA allele combination expressed by the diseased cells in the human patient; wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In various embodiments, the human patient is need of the therapeutic administration. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

In another aspect, provided herein is a pharmaceutical composition for therapeutic administration to a human patient having or suspected of having a pathogen or cancer, comprising: (1) a stimulatory immune checkpoint activator; and (2) a population of human cells comprising antigen-specific T cells that are specific for an antigen of the pathogen or cancer and are derived from a T cell line restricted by a first HLA allele or HLA allele combination expressed by the diseased cells in the human patient; wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen. In various embodiments, the human patient is need of the therapeutic administration. In certain embodiments, the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

When the human patient has a cancer, the diseased cells are the cancerous cells. When the human patient has a pathogen, the diseased cells are the cells infected by the pathogen.

Activity of T cells restricted by the respective HLA allele or HLA allele combination based on recognition of the antigen can be measured by using T cell lines generated in the same way, for example, generated by a method described in Section 5.4.

A T cell line may contain cells other than T cells. However, preferably, a T cell line is enriched for T cells (e.g., comprises more than 70%, more than 75%, more than 80%, more than 85%, more than 90%, more than 95%, or more than 99% T cells). A T cell line can be a collection of primary cells or a collection of cultured cells. Cells in the T cell line can be developed from a single cell or from multiple cells. In various embodiments, a T cell line is derived from a single human donor.

In specific embodiments of the pharmaceutical composition for therapeutic administration to a human patient, the first HLA allele or HLA allele combination is classified as a subdominant HLA allele or HLA allele combination based on being associated with an indication of lower activity based on recognition of the antigen that is lower than the relative activity associated with a second HLA allele or HLA allele combination expressed by the diseased cells in the human patient according to a Representation of Activity, which Representation of Activity (i) identifies a plurality of HLA alleles and optionally HLA allele combinations, and (ii) discloses indications of relative activities of T cell lines, each recognizing at least one epitope of an antigen, and restricted by different ones of the HLA alleles or HLA allele combinations in the plurality; wherein in the Representation of Activity each identified HLA allele or HLA allele combination is associated with the respective indication of relative activity of the T cell line restricted by the HLA allele or HLA allele combination, the relative activities being relative measures of known activity of the T cell lines based on recognition of the antigen. In a specific embodiment, the first HLA allele or HLA allele combination is associated with an indication of no activity based on recognition of the antigen according to the Representation of Activity. An indication of no activity based on recognition of the antigen can be no detectable activity as measured in the generation of the Representation of Activity, or can be an indication of activity that is detectable but is deemed by one of ordinary skill in the art to be indicative of no clinical efficacy. In certain embodiments, a T cell line restricted by the second HLA allele or HLA allele combination is available and is suitable for therapeutic administration to the human patient.

As discussed above, in one embodiment, an HLA allele or HLA allele combination is determined to be subdominant based on comparisons among HLA alleles and HLA allele combinations that are suitable for therapeutic administration to the human patient. T cells are suitable for therapeutic administration to the human patient when a T cell line restricted by the respective HLA allele or HLA allele combination is available and is suitable for therapeutic administration. For example, if a T cell line is observed to have no or too few viable cells in the cell line sample (or after the cell line sample is thawed after freezing), the T cell line is unsuitable for therapeutic administration. As but another example, if the relative activities in the Representation of Activity are based upon in vitro or ex vivo assays of activity and it is known that the relative in vivo activity of a T cell line restricted by a particular HLA allele or HLA allele combination does not correlate with the relative in vitro or ex vivo assay used for generating the Representation of Activity, such that the highest relative activity in the Representation of Activity is not the highest relative in vivo activity, the particular HLA allele or HLA allele combination (by which such T cell line is restricted) can be deemed unsuitable for therapeutic administration. For example, it has been observed that the in vivo activity against CMV infection in human patients for T cell lines restricted by HLA-B35 is clinically ineffective (therefore negligible relative in vivo activity), although the percentage of interferon-γ-secreting CD3+ T cells derived from T cell lines restricted by HLA-B35 indicates a much higher relative activity; thus, in the context of treating CMV infections, in a specific embodiment, a T cell line restricted by HLA-B35 is deemed unsuitable for therapeutic administration. In a related specific embodiment, a T cell line restricted by a particular HLA allele or HLA allele combination is deemed unsuitable for therapeutic administration if T cell line(s) restricted by the particular HLA allele or HLA allele combination are known to be clinically ineffective in treatment of human patients having the pathogen or cancer. In alternative specific embodiments, however, a T cell line restricted by a particular HLA allele or HLA allele combination is not deemed as unsuitable if T cell line(s) restricted by the particular HLA allele or HLA allele combination are known to be clinically ineffective in treatment of human patients having the pathogen or cancer (thus, in the context of treating CMV infections, a T cell line restricted by HLA-B35 is not deemed unsuitable for therapeutic administration in these other specific embodiments), since the combination therapy of the invention may augment efficacy of such T cell line.

In specific embodiments of the aspects and embodiments described herein, the epitope of the antigen that is bound to the HLA protein encoded by the first HLA allele has the highest binding affinity for the first HLA allele among all epitopes of the antigen. In a specific embodiment, the epitope has the highest binding affinity for the first HLA allele among all epitopes of all antigens that can be presented by the first HLA allele.

In some embodiments of the aspects and embodiments described herein, the activity of T cells (and the relative activities of T cell lines, when a Representative of Activity is used) is in vitro antigen reactivity (for example, cytotoxic activity) of the T cells (or T cell lines, as the case may be) against the antigen (for example, against cells expressing the antigen). The in vitro antigen reactivity (for example, cytotoxic activity) of the T cells (or T cell lines, as the case may be) can be measured as described in Section 5.4.1. In other embodiments of the aspects and embodiments described herein, the activity of T cells (and the relative activities of T cell lines, when a Representative of Activity is used) is in vivo clinical efficacies of the T cells (or T cell lines, as the case may be) in treatment of human patients having the pathogen or cancer.

The pharmaceutical composition or pharmaceutical composition for therapeutic administration to a human patient, as described herein, is derived from a T cell line that preferably (1) exhibits substantial antigen reactivity (for example, cytotoxicity) toward fully or partially HLA-matched (relative to the human donor of the T cell line) antigen presenting cells that are loaded with or genetically engineered to express one or more peptides or proteins derived from the antigen of the pathogen or cancer; (2) lacks substantial alloreactivity; and/or (3) is restricted by an HLA allele or HLA allele combination shared with the diseased cells in the human patient, and/or shares at least 2 HLA alleles (e.g., at least 2 out of 8 HLA alleles) with the diseased cells in the human patient. Thus, preferably, antigen reactivity (for example, cytotoxicity), alloreactivity, information as to which HLA allele(s) the T cell line is restricted, and/or the HLA assignment of the T cell line are measured by a method known in the art before administration to a human patient (for example, such a method as described in Koehne et al., 2000, Blood 96:109-117; Trivedi et al., 2005, Blood 105:2793-2801; Haque et al., 2007, Blood 110:1123-1131; Hasan et al., 2009, J Immunol 183: 2837-2850; Feuchtinger et al., 2010, Blood 116:4360-4367; Doubrovina et al., 2012, Blood 120:1633-1646; Doubrovina et al., 2012, Blood 119:2644-2656; Leen et al., 2013, Blood 121:5113-5123; Papadopoulou et al., 2014, Sci Transl Med 6:242ra83; Sukdolak et al., 2013, Biol Blood Marrow Transplant 19:1480-1492; Koehne et al., 2015, Biol Blood Marrow Transplant 21: 1663-1678; or International Patent Application Publication No. WO 2016/073550), as described in Section 5.4.

The pharmaceutical composition or pharmaceutical composition for therapeutic administration to a human patient, as described herein, further comprises a pharmaceutically acceptable carrier.

The pharmaceutical acceptable carrier can be any physiologically-acceptable solution suitable for the storage and/or therapeutic administration of T cells and the inhibitory immune checkpoint inhibitor or the stimulatory immune checkpoint activator.

Also provided herein are kits comprising in one or more containers the pharmaceutical composition described above.

Optionally associated with such one or more containers can be a notice in the form prescribed by a governmental agency regulating the manufacture, use or sale of pharmaceuticals or biological products, which notice reflects approval by the agency of manufacture, use or sale for human administration.

The pharmaceutical compositions and kits encompassed herein can be used in accordance with the methods of treating a human patient as provided in this disclosure.

5.6. Immune Checkpoint Modulators

5.6.1. Inhibitory Immune Checkpoint Inhibitors

The inhibitory immune checkpoint inhibitor can be any pharmaceutical agent that inhibits or blocks the activity of an inhibitory immune checkpoint molecule. In specific embodiments, the activity is binding to the natural binding partner of the inhibitory immune checkpoint molecule. If the inhibitory immune checkpoint molecule is a receptor, the activity can be ligand-binding activity. If the inhibitory immune checkpoint molecule is a ligand, the activity can be receptor-binding activity.

In specific embodiments, the inhibitory immune checkpoint inhibitor is an inhibitor of Programmed Cell Death 1 (PD1), Programmed Death Ligand 1 (PD-L1), Programmed Death Ligand 1 (PD-L2), Cytotoxic T-Lymphocyte Associated Protein 4 (CTLA4), Lymphocyte Activating 3 (LAG3), T-Cell Immunoglobulin And Mucin Domain-Containing Protein 3 (TIM3), V-Domain Ig Suppressor Of T Cell Activation (VISTA), Adenosine A2a Receptor (A2aR), B7 Homolog 3 (B7-H3), B7 Homolog 4 (B7-H4), B and T lymphocyte associated (BTLA), Indoleamine 2,3-Dioxygenase (IDO), Tryptophan 2,3-Dioxygenase (TDO), or Killer-Cell Immunoglobulin-Like Receptor (KIR). In a specific embodiment, the inhibitory immune checkpoint inhibitor is an inhibitor of PD1, PD-L1, PD-L2, CTLA-4, LAG3, TIM-3, VISTA, A2AR, B7-H3, B7-H4, BTLA, IDO, or TDO.

The inhibitory immune checkpoint inhibitor can be an antibody, a small molecule, or an oligonucleotide (such as an aptamer, an shRNA, miRNA, siRNA, or antisense DNA). In specific embodiments, the inhibitory immune checkpoint inhibitor has been approved by Food and Drug Administration (FDA) in the United States or a foreign counterpart agency for the treatment of the cancer or a disease caused by the pathogen.

In specific embodiments, the inhibitory immune checkpoint inhibitor is an antibody that binds to and inhibits the activity of the inhibitory immune checkpoint. Antibodies that can be the inhibitory immune checkpoint inhibitor include, but are not limited to, monoclonal antibodies (including Fc-optimized monoclonal antibodies), polyclonal antibodies, multispecific antibodies (e.g., bispecific antibodies), antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments, and fusion proteins containing antibody fragments. In a specific embodiment, the antibody is a monoclonal antibody. Preferably, the antibody is a humanized antibody.

In specific embodiments, the inhibitory immune checkpoint inhibitor is an inhibitor of PD1. In a specific embodiment, the inhibitory immune checkpoint inhibitor is a monoclonal antibody that binds to and inhibits the activity (e.g., ligand-binding activity) of PD1. In a particular embodiment, the monoclonal antibody is nivolumab, pidilizumab, MEDI0680, or pembrolizumab. In a further particular embodiment, the monoclonal antibody is nivolumab. In another specific embodiment, the inhibitory immune checkpoint inhibitor that is an inhibitor of PD1 is AMP-224. In another specific embodiment, the inhibitory immune checkpoint inhibitor that is an inhibitor of PD1 is pidilizumab. In another specific embodiment, the inhibitory immune checkpoint inhibitor that is an inhibitor of PD1 is pembrolizumab. In another specific embodiment, the inhibitory immune checkpoint inhibitor that is an inhibitor of PD1 is MEDI0680. In another specific embodiment, the inhibitory immune checkpoint inhibitor that is an inhibitor of PD1 is STI-A1110. In another specific embodiment, the inhibitory immune checkpoint inhibitor that is an inhibitor of PD1 is TSR-042. In another specific embodiment, the inhibitory immune checkpoint inhibitor that is an inhibitor of PD1 is AUR-012.

In specific embodiments, the inhibitory immune checkpoint inhibitor is an inhibitor of PD-L1. In a specific embodiment, the inhibitory immune checkpoint inhibitor is a monoclonal antibody that binds to and inhibits the activity (e.g., receptor-binding activity) of PD-L1. In a particular embodiment, the monoclonal antibody is mpd13280A, durvalumab, avelumab, bms-936559, or atezolizumab. In another specific embodiment, the inhibitory immune checkpoint inhibitor that is an inhibitor of PD-L1 is RG7446. In another specific embodiment, the inhibitory immune checkpoint inhibitor that is an inhibitor of PD-L1 is STI-A1010.

In specific embodiments, the inhibitory immune checkpoint inhibitor is an inhibitor of B7-H3 (for example, MGA271).

In specific embodiments, the inhibitory immune checkpoint inhibitor is an inhibitor of CTLA4 (for example, ipilimumab).

In specific embodiments, the inhibitory immune checkpoint inhibitor is an inhibitor of LAG3 (for example, BMS-986016).

In a specific embodiment, the inhibitory immune checkpoint inhibitor is MSB-0020718C.

5.6.2. Stimulatory Immune Checkpoint Activators

The stimulatory immune checkpoint activator can be any pharmaceutical agent that activates or promotes the activity of a stimulatory immune checkpoint molecule. In specific embodiments, the activity is binding to the natural binding partner of the stimulatory immune checkpoint molecule. If the stimulatory immune checkpoint molecule is a receptor, the activity can be ligand-binding activity. If the stimulatory immune checkpoint molecule is a ligand, the activity can be receptor-binding activity.

In specific embodiments, the stimulatory immune checkpoint activator is an activator of CD27, CD28, CD40, CD122, CD137, OX40, Glucocorticoid-Induced TNFR-Related Protein Ligand (GITR), or Inducible T-Cell Costimulator (ICOS).

The stimulatory immune checkpoint activator can be, for example, a ligand, a ligand fragment, or a fusion protein containing a ligand of the stimulatory immune checkpoint molecule (when the stimulatory immune checkpoint molecule is a receptor), or an agonist antibody that binds to and activates the activity of the stimulatory immune checkpoint activator. Antibodies that can be the stimulatory immune checkpoint activator include, but are not limited to, monoclonal antibodies (including Fc-optimized monoclonal antibodies), polyclonal antibodies, multispecific antibodies (e.g., bispecific antibodies), antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments, and fusion proteins containing antibody fragments. In a specific embodiment, the antibody is a monoclonal antibody. Preferably, the antibody is a humanized antibody. In specific embodiments, the stimulatory immune checkpoint activator has been approved by Food and Drug Administration (FDA) in the United States or a foreign counterpart agency for the treatment of the cancer or a disease caused by the pathogen.

In specific embodiments, the stimulatory immune checkpoint activator is an activator of OX40 (for example, MEDI0562, or MEDI6383).

In specific embodiments, the stimulatory immune checkpoint activator is an activator of GITR (for example, TRX518 or MK-4166).

In specific embodiments, the stimulatory immune checkpoint activator is an activator of ICOS (for example, GSK3359609).

5.7. Generation of Representation of Activity

The Representation of Activity identifies a plurality of HLA alleles and optionally HLA allele combinations, and discloses indications of relative activities of T cell lines, each recognizing at least one epitope of an antigen of a pathogen or cancer, and restricted by different ones of the HLA alleles or HLA allele combinations in the plurality. In the Representation of Activity, each identified HLA allele or HLA allele combination is associated with the respective indication of relative activity of the T cell line restricted by the HLA allele or HLA allele combination, the relative activities being relative measures of known activity of the T cell lines based on recognition of the antigen.

The relative activities of the T cell lines can be obtained by any in vitro, ex vivo, or in vivo method known in the art.

In preferred embodiments, the relative activities are measured as the in vivo clinical efficacies of the T cell lines in treatment of human patients having the pathogen or cancer. In specific aspects of such embodiments, the relative activities can be measured as the percentage of human patients having or suspected of having the pathogen or cancer that achieve a complete remission (CR) after treatment with the T cell lines. In specific embodiments, the relative activities are measured as the percentage of human patients having or suspected of having the pathogen or cancer that achieve a CR or partial remission (PR) after treatment with the T cell lines.

In some embodiments, the relative activities are measured as the percentage of interferon-γ-producing CD3+ cells derived from each of the T cell lines upon stimulation with antigen presenting cells presenting one or more peptides displaying the antigenicity of the antigen. In specific embodiments, the relative activities are measured by methods modified from or as described in Koehne, G., et al., Blood, 2002. 99: 1730-1740 or Waldrop, S. L., et al., J Clin Invest, 1997. 99: 1739-1750.

In some embodiments, the relative activities are measured as the percentage of cells expressing an antigen of the pathogen or cancer that are lysed upon exposure to each of the T cell lines in a cytotoxicity assay, carried out according to methods known in the art, for example, a method described in Section 5.4.1.

According to the present invention, the relative activities are not measured as the binding affinities of the epitope recognized by the respective T cell line to the HLA protein that presents the epitope.

In some aspects, the Representation of Activity is a list of the plurality of HLA alleles and optionally HLA allele combinations ranked by the relative activities. In some embodiments, the T cell line for therapeutic use is selected by going down the list of the plurality of HLA alleles and optionally HLA allele combinations ranked by the relative activities, with the highest rank in the list being an indication of the highest relative activity, and determining an HLA allele or HLA allele combination that is not the highest in rank (and, if the T cell line selected is for use in a particular human patient, said HLA allele or HLA allele combination is not the highest in rank among HLA alleles or HLA allele combinations expressed by the diseased cells in the human patient), and choosing a T cell line restricted by that determined HLA allele or HLA allele combination. By way of example, in a specific embodiment, the Representation of Activity is a list as shown in Table 6 of Example 2.

In some aspects, the Representation of Activity is a database (e.g., table) listing the plurality of HLA alleles and optionally HLA allele combinations, each associated with a score indicative of relative activity. In some embodiments, the T cell line for therapeutic use is selected by going through the database listing of the plurality of HLA alleles and optionally HLA allele combinations, each associated with a score indicative of relative activity, with the highest score in the database being an indication of the highest relative activity, and determining an HLA allele or HLA allele combination that does not have the highest score (and, if the T cell line selected is for use in a particular human patient, said HLA allele or HLA allele combination does not have the highest score among HLA alleles or HLA allele combinations expressed by the diseased cells in the human patient), and choosing a T cell line restricted by that HLA allele or HLA allele combination. In a specific embodiment, the selection of a T cell line using a Representation of Activity, that is such a database, can be carried out by first filtering out (excluding) all the HLA alleles and HLA allele combinations in the database that are not expressed by the diseased cells in the human patient, and then determining among those remaining, the HLA allele or HLA allele combination associated with an indication of relative activity that is not the highest, and then choosing a T cell line restricted by that determined HLA allele or HLA allele combination.

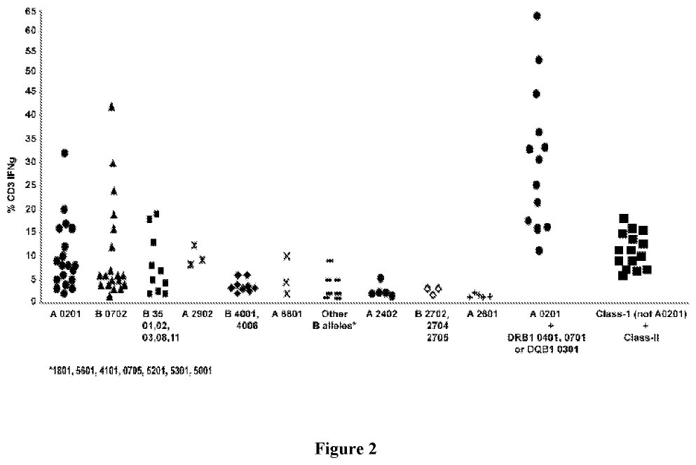

In some aspects, the Representation of Activity is a scatter plot. In certain embodiments, a first axis of the scatter plot represents different ones of the HLA alleles and optionally HLA allele combinations in the plurality of HLA alleles and optionally HLA allele combinations. In certain embodiments, a second axis of the scatter plot represents relative activities. In a specific embodiment, the second axis of the scatter plot represents percentage of interferon-γ-secreting CD3+ cells derived from each T cell line for which an indication of relative activity is disclosed in the Representation of Activity, upon stimulation with antigen presenting cells presenting one or more peptides displaying the antigenicity of the antigen. In a particular embodiment, the stimulation is with antigen presenting cells that are autologous to the respective T cell line and are loaded with one or more peptides displaying the antigenicity of the antigen, as the indication of said relative activity. By way of example, in a specific embodiment, the Representation of Activity is a scatter plot as shown in FIG. 2.

In some embodiments, the Representation of Activity is stored in a database.

In various embodiments, the method of selecting a T cell line is computer-implemented. In some embodiments, the method of selecting a T cell line is computer-implemented using a computer system as described in Section 5.9. In some embodiments, the method of selecting a T cell line is computer-implemented using a computer readable medium as described in Section 5.9.

Additional data can be used to update a Representation of Activity once the additional data is available.

5.8. Generation of Representation of Frequency

The Representation of Frequency identifies a plurality of HLA alleles and optionally HLA allele combinations, and discloses indications of relative frequencies of generation of T cell lines, each recognizing at least one epitope of an antigen of a pathogen or cancer, and restricted by different ones of the HLA alleles. In the Representation of Frequency, each identified HLA allele or HLA allele combination is associated with the respective indication of relative frequency of generation of the T cell lines restricted by the respective HLA allele or HLA allele combination.

In some aspects, the Representation of Frequency is a list of the plurality of HLA alleles and optionally HLA allele combinations ranked by the relative frequencies, and the highest rank is an indication of the highest frequency of generation.

In some aspects, the Representation of Frequency is a database (e.g., table) listing the plurality of HLA alleles, each associated with a score indicative of relative frequency, and the highest score is an indication of the highest frequency of generation.

In some embodiments, the Representation of Frequency is stored in a database.

In various embodiments, the method of selecting a T cell donor as described in this disclosure is computer-implemented. In some embodiments, the method of selecting a T cell donor as described in this disclosure is computer-implemented using a computer system as described in Section 5.9. In some embodiments, the methods of selecting a T cell donor as described in this disclosure is computer-implemented using a computer readable medium as described in Section 5.9.

Additional data can be used to update a Representation of Frequency once the additional data is available.

5.9. Computer Systems and Computer Readable Media

In various embodiments, a computer system or computer readable medium is configured for carrying out any of the methods of selecting a T cell line, and any of the methods of selecting a T cell donor as described in this disclosure.

Also provided herein are computer systems for selecting a T cell line for therapeutic administration to a human patient having or suspected of having a pathogen or cancer. In a specific embodiment such a computer system comprises: a central processing unit; a memory, coupled to the central processing unit, the memory storing instructions for performing the step(s) of any of the methods of selecting a T cell line or any of the methods of selecting a T cell donor as described in this disclosure. In some embodiments, the computer system further comprises a display device in operable communication with the central processing unit.

Also provided herein are computer readable media having computer-executable instructions for performing the step(s) of any of the methods of selecting a T cell line or any of the methods of selecting a T cell donor as described in this disclosure.

In some embodiments, loaded into a computer system or computer readable medium are software components that are standard in the art. The software components collectively cause the computer system to function according to a method of selecting a T cell line or the method of selecting a T cell donor as described in this disclosure. In some embodiments, loaded into the computer system or computer readable medium are software components that are standard in the art, and one or more computer program products that are special to the instant invention. In specific embodiments, the one or more computer program products cause a computer system to function according to a method of selecting a T cell line or the method of selecting a T cell donor as described in this disclosure. In specific embodiments, the one or more computer program products that are special to the instant invention and the software components that are standard in the art collectively cause the computer system to function according to a method of selecting a T cell line or the method of selecting aT cell donor as described herein.

5.10. Antigen Specificity and Patients

The antigen of a pathogen or cancer can be a peptide or protein whose expression is higher in the diseased cells (for example, cells infected by the pathogen, or cancerous cells) relative to non-diseased cells (for example, cells not infected by the pathogen, or non-cancerous cells), or a peptide or protein that is uniquely expressed in the diseased cells (for example, cells infected by the pathogen, or cancerous cells) relative to non-diseased cells (for example, cells not infected by the pathogen, or non-cancerous cells).

In some embodiments, the antigen is an antigen of a pathogen. In various embodiments, the human patient has the pathogen. The pathogen can be a virus, bacterium, fungus, helminth or protist.

In specific embodiments, the pathogen is a virus. In a specific embodiment, the virus is cytomegalovirus (CMV). In an aspect of the specific embodiment, the antigen of CMV is CMV pp65 or CMV IE1. In another aspect of the specific embodiment, the antigen of CMV is CMV pp65. In another specific embodiment, the virus is Epstein-Barr virus (EBV). In an aspect of the specific embodiment, the antigen of EBV is EBNA1, EBNA2, EBNA3A, EBNA3B, EBNA3C, LMP1, or LMP2. In another aspect of the specific embodiment, the antigen of EBV is EBNA1, LMP1, or LMP2. In another specific embodiment, the virus is BK virus (BKV), John Cunningham virus (JCV), herpesvirus (such as human herpesvirus-6 or human herpesvirus-8), human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

In specific embodiments, the pathogen is a bacterium, such as a mycobacterium or Chlamydia trachomatis. In specific embodiments, the pathogen is a fungus, such as Cryptococcus neoformans, Pneumocystis jiroveci, a Candida, or an invasive fungus. In specific embodiments, the pathogen is a helminth. In specific embodiments, the pathogen is a protist, such as Toxoplasma gondii. In specific embodiments, the pathogen is a protozoa.

In specific embodiments, the human patient has an infection of the pathogen. In a specific embodiment, the pathogen is CMV and the human patient has a CMV infection (e.g., CMV viremia, CMV retinitis, CMV pneumonia, CMV hepatitis, CMV colitis, CMV encephalitis, CMV meningoencephalitis, CMV-positive meningioma, or CMV-positive glioblastoma multiforme). In another specific embodiment, the pathogen is EBV and the human patient has an EBV-positive lymphoproliferative disorder (EBV-LPD) (for example, an EBV-positive post-transplant lymphoproliferative disorder) resulting from EBV infection, such as B-cell hyperplasia, lymphoma (such as, B-cell lymphoma, non-Hodgkin lymphoma (e.g., diffuse large B-cell lymphoma, for example in the elderly), T-cell lymphoma, EBV-positive Hodgkin's lymphoma, Burkitt lymphoma), polymorphic or monomorphic EBV-LPD, autoimmune lymphoproliferative syndrome, or mixed PTLD (post-transplant lymphoproliferative disorder). In another specific embodiment, the pathogen is EBV and the human patient has an EBV-positive nasopharyngeal carcinoma. In another specific embodiment, the pathogen is EBV and the human patient has an EBV-positive gastric cancer. In another specific embodiment, the pathogen is EBV and the human patient has an EBV-positive leiomyosarcoma. In another specific embodiment, the pathogen is EBV and the human patient has an EBV-positive NK/T lymphoma. In another specific embodiment, the pathogen is EBV and the human patient has an EBV viremia.

In other embodiments, the antigen is an antigen of a cancer. In various embodiments, the human patient has the cancer. In certain embodiments, the antigen is Wilms Tumor 1 (WT1).

The cancer can be a blood cancer, such as, but is not limited to: acute lymphoblastic leukemia, chronic lymphocytic leukemia, acute myelogenous leukemia, chronic myelogenous leukemia, hairy cell leukemia, T-cell prolymphocytic leukemia, Large granular lymphocytic leukemia, adult T-cell leukemia, plasma cell leukemia, Hodgkin lymphoma, Non-Hodgkin lymphoma, or multiple myeloma. In a specific embodiment, the cancer is multiple myeloma or plasma cell leukemia. In an aspect of the specific embodiment, the antigen of the cancer is WT1.

The cancer can also be a solid tumor cancer, including, but is not limited to, a sarcoma, a carcinoma, a lymphoma, a germ cell tumor, or a blastoma. The solid tumor cancer that can be, such as, but is not limited to: a cancer of the breast, lung, ovary, stomach, pancreas, larynx, esophagus, testes, liver, parotid, biliary tract, colon, rectum, cervix, uterus, endometrium, kidney, bladder, prostate, thyroid, brain, or skin.

In a specific embodiment, the human patient is an adult (at least age 16). In another specific embodiment, the human patient is an adolescent (age 12-15). In another specific embodiment, the patient is a child (under age 12).

In a specific embodiment, the human patient has failed a previous therapy for the pathogen or cancer, which previous therapy is not treatment with a population of human cells comprising antigen-specific T cells in combination with an inhibitory immune checkpoint inhibitor or a stimulatory immune checkpoint activator, due to resistance to or intolerance of the previous therapy. A disease is considered resistant to a therapy, if it has no response, or has an incomplete response (a response that is less than a complete remission), or progresses, or relapses after the therapy. The previous therapy could be an antiviral agent known in the art (e.g., an antiviral drug or antibody), or an anti-cancer therapy known in the art (e.g., a chemotherapy or a radiotherapy), as the case may be.

### 6. EXAMPLES

Certain embodiments provided herein are illustrated by the following non-limiting examples.

### 6.1. Example 1: Adoptively Transferred CMV-Specific T Cells Restricted by Dominant and Subdominant HLA Alleles in Combination with PD1 Inhibition Demonstrate Improved In Vivo Inhibition of Tumor Xenografts

Adoptive transfer of transplant donor or third party donor derived CMV-specific T cells (CMV-CTL) can effectively treat CMV infections in HSCT recipients. In clinical trials, infusion of partially matched third party CMV-CTLs, has demonstrated high response rates against persistent CMV infection. T cells generated in vitro or directly selected in vivo demonstrate a striking preponderance of specificity for 1-2 immunodominant epitopes presented by specific HLA alleles. Immunodominant HLA alleles are associated with higher T cell functional activity in vivo, compared to subdominant HLA alleles. Agents augmenting activity of T cells restricted by subdominant HLA alleles have not been explored.

When HLA-A0201 and HLA-A2402 are co-inherited in humans, HLA-A0201 is immunodominant over HLA-A2402, and thus preponderance of the responsive T cells are directed to HLA-A0201 rather than to HLA-A2402. We used an in vivo model to assess efficacy of CMV-CTLs using colon carcinoma cells (coca) transduced to express CMVpp65 as a surrogate system. HLA-A0201⁺ and HLA-A2402⁺ human colon carcinoma cells were transduced to express CMVpp65 and GFP-firefly luciferase (cocapp65). CMV-CTLs responding to either the immunodominant HLA-A0201 presented NLV epitope (A2-NLV) or the subdominant HLA-A2402 presented QYD epitope (A24-QYD) were generated from donors co-inheriting HLA-A0201 and HLA-A2402 by in vitro stimulation using NIH 3T3 artificial antigen presenting cells, expressing HLA-A0201 or HLA-A2402, B7.1, LFA-3 and ICAM1. Tumor cells (10⁵ cells) were injected subcutaneously into groups of 5-6 NOD/Scid-IL2Rγc^{-/-} mice (NSG mice) on the right flank, and 10⁵ cells from a pp65 expressing melanoma cell line (melpp65), lacking expression of HLA-A0201 or HLA-A2402, were injected on the left shoulder as control. Two groups each received 10⁶ of tetramer⁺ A2-NLV or A24-QYD CMV-CTLs intravenously per mouse; one of each CMV-CTL treated group also received 2 intravenous doses (200 μg/dose) of anti-PD1 antibody (nivolumab) at day 2 and 7 post CTL infusion. Control groups received IL-2, with or without anti-PD1, or HLA mismatched CMV-CTLs. Tumor growth was monitored by bioluminescent imaging.

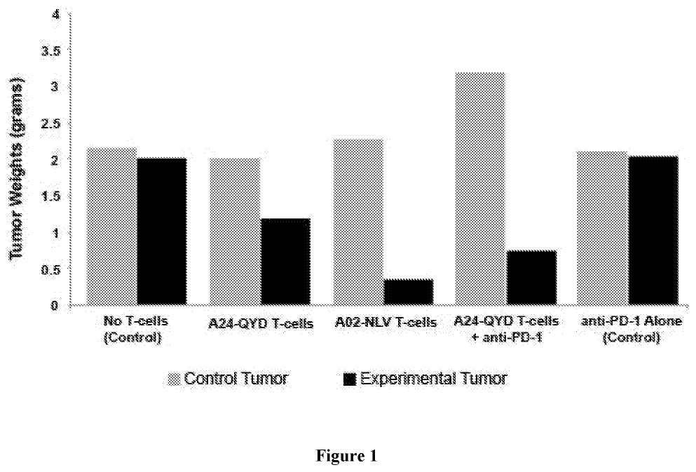

CMV-CTLs responsive to the subdominant HLA-A2402 presented QYD epitope induced significant cocapp65 growth suppression compared to controls, but did not eradicate tumors in any animal. Combined treatment of A24-QYD CMV-CTLs with anti-PD1 antibody induced complete cocapp65 eradication in 2 of 5 mice, with minute residual tumors in 3 mice. Treatment with the immunodominant HLA-A0201-restricted CMV-CTLs induced complete cocapp65 eradication in 2 of 5 mice, and smaller residual tumors compared to subdominant HLA-A2402 restricted CTL treatment. Combined treatment with anti-PD1 and A2-NLV CMV-CTLs led to complete cocapp65 eradication in 3 of 5 mice, with minute tumors in 2 mice. Tumor weights were measured (see FIG. 1). Taken together, these data provide evidence that blocking the PD1/PD-L1 interaction can synergistically augment the antiviral activity of both immunodominant and subdominant HLA allele-restricted CMV-CTLs.

### 6.2. Example 2: Establishment of HLA Allele Hierarchy

Example 2 is essentially as described in International Patent Application Publication No. WO 2016/073550.

6.2.1. Methods:

6.2.1.1. Establishment of CMV CTL Bank:

All cellular products were processed in the GMP facility at Memorial Sloan Kettering Cancer Center (MSKCC) under standard operating procedures (SOPs) and FDA compliant protocols.

6.2.1.1.1. Generation of Autologous Cytokine-Activated Monocytes (CAMS):

Peripheral blood mononuclear cells (PBMC) were isolated from the blood of seropositive donors by density gradient centrifugation using ficoll hypaque.

PBMC at a concentration of 10⁷/ml suspended in RPM-1640 with 1% autologous serum were allowed to adhere in 6 well tissue culture plates at 37° C. for 2 hours following which the non-adherent mononuclear cells were gently removed. The adherent monocytes cultured with 2 ml serum free IMDM per well and supplemented with GM-CSF 2000 IU (50 μl) and IL-4 1000 U (25 μl) of IL-4 every other day until day 5. On day 5, tumor necrosis factor-α (SIGMA, St. Louis) was added to achieve a final concentration of 10 ng/ml, Interleukin-1β to 400 IU/ml, interleukin-6 (R&D systems, Inc, Minneapolis, Minn. USA) to 1000 IU/ml and prostaglandin-E2 (Calbiochem, La Jolla, Calif. USA) to 25 mm/ml to induce final maturation of the CAMS. On day 7 the mature CAMS were harvested, characterized as to their expression of HLA Class II, CD14 and co-stimulatory molecules by FACS counted, aliquoted and used for sensitization of T-cell lines as detailed below.

6.2.1.1.2. Generation of Autologous Transformed B Lymphocyte Cell Lines (BLCL):

EBV-BLCLs from each donor were generated by infections of PBMC with EBV strain B95.8 as previously described (Koehne, G., et al., Blood, 2000. 96: 109-117; Koehne, G., et al., Blood, 2002. 99: 1730-1740). The cells were maintained in RPMI 1640 (Invitrogen, Inc, Carlsbad, Calif. USA) supplemented with 10% fetal calf serum (FCS), and acyclovir.

6.2.1.1.3. Generation of CMVpp65 Specific T-Cells:

T-cells were enriched from peripheral blood lymphocytes separated from the PBMCs by depletion of adherent monocytes followed by depletion of natural killer cells by using immunomagnetic separation of CD56+ cells with immunomagnetic CD56 precoated microbeads (Miltenyi Biotech Inc.). Purified T-cells were then co-cultured with irradiated autologous CAMS loaded with a GMP grade pool of overlapping pentadecapeptides (PL CAMs) as previously described (Trivedi, D., et al., Blood, 2005. 105: 2793-2801). T-cells were cultured for a period of 28-40 days in the presence of IL-2 (5-40 U/ml), and re-stimulated weekly with irradiated autologous peptide-loaded CAMS, at an effector to stimulator ratio of 20:1 as previously described (Trivedi, D., et al., Blood, 2005. 105: 2793-2801).

6.2.1.2. Characterization of CMVpp65 Specific T-Cells:

6.2.1.2.1. Tetramer Analysis:

The proportion of CMVpp65 epitope specific T-cells were quantitated using HLA-peptide tetramers using commercially available CMVpp65 MHC-peptide tetramers for HLA A0201, A2402 and B0702 bearing peptide sequences NLVPMVATV, QYDPVAALF and TPRVTGGGAM respectively (Beckman Coulter, Inc Fullerton, Calif.). T-cells were incubated with CD3 FITC, CD8 PE, CD4 PerCP (BD Bioscience, San Jose, Calif.) and APC conjugated tetrameric complex for 20 minutes on ice, washed and subsequently analyzed by FACS (BD LSR II). Data were analyzed using Flowjo software (Tree Star Inc, Ashland, Oreg.). The proportion of CD4 and CD8+ T-cells within the cultures, as well as the proportion of CD3+, and CD8+ T-cells binding to the HLA-peptide tetramers was determined.

6.2.1.2.2. TCR Vβ Repertoire

CMV peptide-HLA tetramer+ T-cells were analyzed for TCRVβ repertoire via flow cytometry using commercially available kit containing antibodies to 24 subfamilies of the Vβ region of the human TCR (TO Test® Beta Mark, Beckman Coulter, Inc, France) according to procedures provided by the manufacturer (Wei, S., et al., Immunogenetics, 1994. 40: 27-36).

6.2.1.2.3. Quantitation of CMV-Specific and Alloreactive IFN-γ-Producing T Cells

At the onset and at several points in the development of each CMV-specific T-cell line, donor T lymphocytes at a concentration of 1×10⁶/mL were mixed with autologous CAMS that were loaded with the pool of CMVpp65 peptides (20 ug/ml) at an effector-stimulator cell ratio of 5:1. Control tubes containing effector cells and PBMCs not loaded with any peptide were set up in parallel. Brefeldin A was added to nonstimulated and peptide stimulated samples at a concentration of 10 μg/mL cells. Tubes were incubated overnight for 16 hours in a humidified 5% CO₂ incubator at 37° C.

Aliquots of the bulk nonstimulated and of the stimulated cultures were transferred to tubes for staining with monoclonal antibodies. Cells were stained with 5 μL monoclonal anti-CD3 labeled with allophycocyanin (APC) and 10 μL anti-CD8 peridin chlorophyll protein (PerCP) or anti-CD4 PerCP (BD Biosciences, San Jose, Calif.) and were incubated for 20 minutes at room temperature in the dark. Cells were washed with 2 mL phosphate-buffered saline (PBS)-bovine serum albumin (BSA)-azide (AZ) (PBS+0.5% BSA+0.1% AZ). Cells were centrifuged, supernatant discarded, and 100 μL reagent A (Fix & Perm Cell Permeabilization Reagents A & B; Caltag Laboratories, Burlingame, Calif.) was added to each tube to fix the cells. These cells were then incubated for 15 minutes. Cells were washed with PBS+ BSA+AZ, and 100 μL reagent B (Caltag Laboratories) was added for permeabilization. Intracellular staining was performed by adding 10 μL mouse IgG1 isotype control fluorescein isothiocyanate (FITC) or IFN-γ FITC (BD PharMingen, San Diego, Calif.) monoclonal antibody. Cells were incubated for 20 minutes at room temperature, in the dark, washed twice, and further fixed in 1% formalin.

Stained and fixed cells were subsequently acquired using an LSR II flow cytometer with three lasers for 10-color capability (BD Biosciences), and analyzed using flowjo software. Cells were first identified by forward and side light scatter and then by gating the CD3+ cells in a CD3 APC versus side scatter dot plot. Twenty to Fifty thousand events were acquired in the combined gate. For further identification of the cells, gating on the CD3+CD8+ or CD3+CD4+ cells was performed. Quadrant markers were established based on analysis of the nonstimulated control and isotype control tubes.

6.2.1.3. Quantitating Antiviral CD8+ T-Cell Responses to Different CMVpp65 Epitopes

6.2.1.3.1. Epitope Mapping Using a Library of Overlapping 15 aa Peptides

T-cell responses to specific peptides within CMV pp65 were identified and quantitated by measuring the number of IFNγ positive T-cells generated upon secondary stimulation with autologous APCs loaded with the peptides or peptide pool (PL) of interest, according to the technique of Waldrop et al (Waldrop, S. L., et al., J Clin Invest, 1997. 99: 1739-1750) as modified by Koehne et al (Koehne, G., et al., Blood, 2002. 99: 1730-1740). A grid of overlapping peptide pools permitted the identification of specific epitopes inducing T-cell responses. Peptide-loaded PBMCs that were autologous to the T cell donor, CAMS that were autologous to the T cell donor, or BLCLs that were autologous to the T cell donor were used as APCs to stimulate the responding T-cells for epitope mapping.

6.2.1.3.2. In-Vitro Cytotoxic Activity

All T-cells lines were assessed for their capacity to lyse CMVpp65 loaded targets using a standard ⁵¹chromium release assay as previously described (Koehne, G., et al., Blood, 2002. 99: 1730-1740; Trivedi, D., et al., Blood, 2005. 105: 2793-2801). Targets used in all experiments consisted of a panel of EBV-BLCL, each sharing with T-cells of a given donor a single HLA allele. These cells were loaded, as specified for a given experiment, with the complete pool of CMVpp65 peptides, or specific sub-pools thereof, single pentadecapeptides, or a CMV pp65 nonamer known to be presented by that allele (e.g. NLVPMVATV for HLA A0201, QYDPVAALF for HLA A2402 and TPRVTGGGAM and RPHERNGFTV for HLA B0702) (Trivedi, D., et al., Blood, 2005. 105: 2793-2801; Hasan, A. N., et al., J Immunol, 2009. 183: 2837-2850). Targets loaded with peptides not presented by the shared HLA allele were used as controls. HLA restriction was identified by reactivity against targets pulsed with an identified peptide epitope presented on a specific shared HLA allele, and absence of reactivity against peptide loaded on either EBV BLCL bearing other shared alleles or fully mismatched EBV BLCL.

6.2.2. Data:

6.2.2.1. GMP Grade CMV CTL Bank Generated from a Genotypically Heteregeneous Donor Population Inheriting a Diverse Array of HLA Alleles

A total of 119 CMVpp65 specific CTL lines have been generated over a span of 7 years since the initiation of the clinical trial using donor derived CMVpp65 specific T-cells for treatment of CMV viremia in recipients of allogeneic HSCT.

The pool of donors used for the generation of the CTL lines inherited 180 different HLA alleles which were representative of the common HLA alleles prevalent in the multiethnic population of New York. The distribution of the HLA alleles in the donor CTL pool also closely correlated with the HLA allele frequencies represented in each of the ethnic populations including Caucasian, Asian and blacks, except for HLA A0201 and B0702, which were over represented; 33% vs 25% and 21% vs 8.7% respectively (Table 1). The order of the frequency of inherited HLA class-I alleles among the 119 donors was as follows: A0201 (n=39), A0301 (n=28), B0702 (n=25), B 44 (n=24), HLA B 0801 (n=22), B 3501-11 (n=19), A1101 (n=16), A2402 (n=14), B 1501-17 (n=14), B 1801-07 (n=12), A3201-03 (n=11), A3301-04 (n=10), B 4001-06 (n=9), and A2601 (n=9), B 5701 (n=9). Other HLA class-I alleles were represented at lower frequencies, such as HLA B 5201 (n=8), B 3801 (n=6), A6801-09 (n=5), B 5801 (n=5). For HLA class-II alleles, there were 6 HLA DRB1 alleles that were highly represented, as expected from their higher frequencies in the general population (Table 1.). In order of frequency, these included DRB1 1501-08, 0401-32, 0301-13, 0701-04, 1101-20, 1301-34.

TABLE 1
HLA allele frequencies in general population and characterization
of 119 CMVpp65 specific CTL lines.

| | | | | CTL Lines = 119 | | |
|---|---|---|---|---|---|---|
| | | | | | HLA Allele | |
| | | | | CTL | Restricting | |
| | HLA Allele Frequency | | | Lines | T cell Cytotoxic | |
| | in General Population | | | Inheriting | Response | |
| HLA Allele | Cauc. | Black | Oriental | Allele | N | % CTL Lines |
| A0101 | 14.07 | 4.85 | 3.66 | 23 | 3 | 12 |
| A0201 | 25.01 | 15.75 | 3.22 | 39 | 32 | 82 |
| A0301 | 11.9 | 6.48 | 3.23 | 29 | 0 | 0 |
| A1101 | 6.87 | 1.45 | 16.33 | 16 | 1 | 6.2 |
| A2301-05 | 2.5 | 11.77 | 0.8 | 4 | 0 | 0 |
| A2402-07 | 10.3 | 3.14 | 23.97 | 16 | 3 | 18.7 |
| A2501 | 2.12 | 0.45 | 0.46 | 2 | 0 | 0 |
| A2601 | 4.22 | 3.33 | 3.85 | 9 | 4 | 44.4 |
| A2901-02 | 3.01 | 3.94 | 0.86 | 8 | 2 | 25 |
| A3001-10 | 3.39 | 14.48 | 2.1 | 6 | 1 | 16.7 |
| A3101-10 | 2.52 | 1.88 | 4.62 | 7 | 0 | 0 |
| A3201-10 | 3.92 | 2.03 | 0.62 | 10 | 0 | 0 |
| A3301-10 | 2.72 | 5.72 | 5.13 | 9 | 0 | 0 |
| A6801-02 | 3.99 | 9.68 | 1.29 | 6 | 2 | 33 |
| A6901 | 3.99 | 9.68 | 1.29 | 1 | 0 | 0 |
| A7401-09 | | | | 2 | 0 | 0 |
| B0702 | 8.67 | 7.71 | 3.37 | 25 | 25 | 100 |
| B0801 | 7.41 | 4.83 | 1.4 | 22 | 1 | 4.5 |
| B1301-09 | 3.12 | 1.05 | 7.45 | 3 | 0 | 0 |
| B1401-09 | 3.29 | 3.45 | 0.68 | 7 | 0 | 0 |
| B1501-09 | 4.06 | 0.92 | 8.43 | 13 | 0 | 0 |
| B1801-09 | 6.31 | 4.62 | 0.92 | 11 | 1 | 9 |
| B2701-05 | 3.71 | 1.46 | 3.62 | 5 | 2 | 40 |
| B3501-3511 | 10.33 | 5.53 | 5.03 | 19 | 9 | 47.4 |
| B3801 | 2.41 | 0.35 | 2.1 | 5 | 0 | 0 |
| B4001-4006 | 3.12 | 0.45 | 9.03 | 11 | 2 | 18.2 |
| B4201-02 | 0.14 | 5.06 | 0.06 | 3 | 3 | 100 |
| B4401-03 | 11.19 | 5.75 | 3.59 | 22 | 4 | 18.2 |
| DRB1 0301 | 11.1 | 13.99 | 5.02 | 27 | 5 | 18.5 |
| DRB1 | 12.82 | 10.51 | 12.99 | 23 | 4 | 17.4 |
| 0401-04 | | | | | | |
| DRB1 0701 | 13.17 | 9.23 | 5.77 | 28 | 3 | 10.7 |
| DRB1 | 13.36 | 15.74 | 7.74 | 26 | 8 | 30.8 |
| 1101-04 | | | | | | |
| DRB1 | 10.73 | 9.91 | 14.35 | 31 | 5 | 16 |
| 1501-02 | | | | | | |

6.2.2.2. CMVpp65 Specific T-Cell Responses are Dominated by Epitopes Presented by a Limited Number of HLA Class-I and Class-II Alleles

In 103 of the 119 (87%) CTL lines, the immunodominant T-cell responses were restricted by HLA class-I alleles, and in 16 CTL lines, by HLA class-II alleles. In 54% of the CTL lines, the immunodominant T-cell responses were restricted by 3 HLA Class-I alleles; A0201 (25%), B 0702 (21%) and B3501-11 (8%). Other alleles presenting immunodominant epitopes included HLA A2402, B 4001, B 4006, B 4202, B 4204, B 4402, B 4403, DRB1 0401 and 0404, DRB1 1101, DRB1 1202. Thus, despite the broad array of class-I and class-II HLA alleles represented in this bank, only 19 of these alleles presented epitopes eliciting immunodominant T-cell responses. Furthermore, T-cell responses of any detectable level were specific for epitopes presented by only 49 of the 180 HLA alleles inherited by donors in the bank.

6.2.2.3. The HLA Alleles Presenting Immunodominant Epitopes Exist in a Hierarchical Order within Individuals Co-Inheriting Specific Haplotypes

Evaluation of the T-cell lines in the bank also demonstrated that epitopes presented by specific HLA alleles were consistently dominant, as measured by quantitations of epitope specific IFNγ+ T-cells and ascertainment of their HLA restriction. Previous studies have provided evidence that epitopes of CMVpp65 presented by HLA B0702 are dominant in patients co-inheriting HLA A0201 and B0702 (Lacey, S. F., et al., Hum Immunol, 2003. 64: 440-452). In the series of this example, HLA B0702 was consistently the allele restricting the immunodominant T-cell responses in all 25 donors in the bank inheriting this allele (100%), including 9 that co-inherited HLA A0201. Thus, responses restricted by HLA B0702 were dominant irrespective of the other HLA class-I and class-II alleles inherited.

On the other hand, in 30 of the 39 donors (77%) inheriting HLA A0201, the immunodominant T-cell response was restricted by HLA A0201. The remaining 9 donors were those who co-inherited HLA A0201 and B0702, and in each of these 9 donors, the immunodominant T-cell response was restricted by HLA B 0702. Thus HLA A0201 was the allele restricting the immunodominant T-cell response when co-inherited with any other HLA class-I or class-II alleles, except when co-inherited with HLA B0702. For example, among 22 donors inheriting HLA B44 alleles, only 4 elicited dominant responses restricted by this allele. When these alleles (B4401, B4402, B4403) were co-inherited with HLA A0201, in 11 of 12 such donors (91.6%) the immunodominant CTL responses were restricted by HLA A0201; the other donor also co-inherited HLA B0702 and elicited an HLA B0702 restricted response.

A striking other feature of the T-cell responses in donors inheriting HLA B 0702 or A0201 was the fact that the responses observed were exclusively directed against epitopes presented by these alleles. In contrast, in T cell lines in which responses to immunodominant epitopes were restricted by other HLA alleles, subdominant populations of T-cells specific for other epitopes and restricted by other HLA alleles were commonly observed. This analysis allowed for the recognition of a hierarchical clustering of HLA alleles presenting the immunodominant epitopes as shown in FIG. 2.

The hierarchy of HLA alleles presenting immunodominant epitopes was exclusively based on their level of functional activity in response to peptide stimulation. There was no correlation between the affinity of the peptide for HLA binding and its capacity to elicit immunodominant T-cell responses (Table 2).

TABLE 2
Characterization of HLA alleles presenting immunodominant epitopes.

| | | | | | |
|---|---|---|---|---|---|
| | | | Number | Number | Pedicted |
| | | | of CTL | of CTL | Binding |
| | | Presenting | Lines | Lines Epitope | Score |
| No | Epitope | HLA Allele | Responding | Immunodominant | (SYFPEITHI) |

HLA Class I

| | | | | | |
|---|---|---|---|---|---|
| 1 | NLVPMVATV | A0201 | 31 | 30 | 30 |
| 2 | TPRVTGGGAM | B0702 | 16 | 13 | 19 |
| 3 | RPHERNGFTV | B0702 | 12 | 7 | 17 |
| 4 | HERNGFTVL | B4001 & 4006, | 11 | 10 | 23 |
| | | B4201 & 4202, | | | 23 |
| | | B4403, | | | 23 |
| | | A2601, | | | 8 |
| | | A0101 | | | 4 |
| 5 | EVQAIRETVE | B3501, B3502, | 7 | 5 | 2 |
| | | B3503, B3508, | | | |
| | | B3511 | | | |
| 6 | QYDPVAALF | A2402, 2407 | 5 | 5 | 24 |
| 7 | INVHHYPSAA | A2601 | 3 | 3 | 6 |
| | | A0101 | 1 | 1 | 0 |
| 8 | YSEHPTFTS | B0801 | 4 | 3 | 0 |
| | | A0101 | | | 13 |
| 9 | QMWQARLTV | B5201 | 3 | 2 | not found |
| | | B3502 | 1 | 1 | 1 |
| 10 | VYALPLKMLN | A2402 | 1 | 14 | |
| | | A6801 | 1 | 3 | 5 |
| | | B3501 | 1 | 11 | |
| 11 | FVFPTKDVAL | A2402 | 2 | 2 | 21 |
| | | B3501 | 2 | 2 | 13 |

HLA Class II

| | | | | | |
|---|---|---|---|---|---|
| 11 | EHPTFTSQYRIQG | DRB1 1101, | 8 | 7 | 3 |
| | KL | DRB1 1104, | | | 3 |
| | | DRB1 1501 | | | 4 |
| 12 | KYQEFFWDAND | DRB1 1101, | 6 | 5 | 1 |
| | | DQB1 0501 | | | 18 |
| 13 | QPFMRHERNGF | DRB1 0301 | 3 | 2 | not found |
| | | DRB1 1501 | | | not found |

HLA class I and Class II (Shared)

| | | | | | |
|---|---|---|---|---|---|
| 1 | KYQEFFWDANDI | B1801, | 1 | 1 | not found |
| | YRI | DRB1 1101, | 6 | 5 | 1 |
| | | DQB1 0501 | 1 | 0 | 18 |
| 2 | QIFLEVQAIRETVE | B3501-3511, | 7 | 5 | 2 |
| | | DRB1 1501 | 1 | 0 | 14 |
| 3 | QPFMRHERNGF | A0101, | 1 | 1 | 1 |
| | | B0801 | 1 | 1 | not found |
| | | DRB1 0301 | 2 | 2 | not found |
| | | DRB1 1501 | 1 | 0 | not found |
| 4 | AGILARNLVPMV | A0201 | 31 | 30 | 30 |
| | ATV | DRB1 0401, | 2 | 1 | 14 |
| | | DRB1 0402, | 1 | 1 | 14 |
| | | DRB1 0404 | 1 | 0 | 14 |
| | | DQB1 0301 | 1 | 1 | not found |
| 5 | PQYSEHPTFTSQY | A0101, | 2 | 2 | 13 |
| | RI | B0801 | 2 | 2 | 0 |
| | | DRB1 0301 | 2 | 2 | 0 |

6.2.2.4. The Epitope Repertoire and HLA Alleles Constituting the CMV CTL Bank can be Used for Treatment of a Diverse Patient Population

A very limited repertoire of immunodominant CMVpp65 epitopes eliciting T-cell responses was discovered within the 119 CTL lines constituting the GMP bank, that were presented by a limited number HLA alleles. Given that T-cell responses are defined by such fine specificity; for antigen specific T-cells to be clinically effective in the third party setting, the T-cells selected would need to be responsive to epitopes presented by an HLA allele shared by the patient. Within these parameters, the proportion of ethnically diverse patients that could potentially be treated using CTLs from this bank was analyzed.

A series of consecutive T-cell depleted transplants performed at the Memorial Sloan-Kattering cancer center over the last 3-5 years from donors that were either HLA matched or HLA mismatched related or unrelated, as well as cord blood donors, was reviewed. In a series of 239 HLA matched related or unrelated transplants at the center, in 86% of such cases a CTL line with a CMV T-cell response restricted by an HLA allele shared with the patient and matching at 1-2 additional HLA alleles was able to be identified. Similarly, in a series of 137 HLA mismatched transplants, and 70 cord blood transplants, an appropriately restricted CTL line in 93% and 81% of the cases respectively was able to be identified. Thus, despite the broad representation of HLA alleles in this CTL bank, T-cells restricted by a limited repertoire of HLA alleles could be identified and used for treatment of most patients in this ethnically diverse group.

6.2.2.5. Clinical Activity of the CMV CTLs Selected for Treatment from the Transplant Donor or a Third Party Donor Using the Newly Defined Epitope and HLA Restriction Criteria

A total of 54 evaluable patients received CMV CTLs as treatment of clinical infection or persistent viremia that had failed to respond to antiviral drugs. Of these 19 received CMVpp65-specific T-cells from their HSCT donor (NCTO1646645) and 35 from T-cells from an >2 HLA allele matched third party donor. Results are summarized in Table 3 and Table 4. In this analysis, CR is defined as clearance of clinical infection and/or clearance of detectable CMV from the blood. PR is defined as a reduction of CMV in the blood >2 log 10. SD is defined as patients with stable clinical status and a reduction of CMV of <2 log 10. POD is defined as continued progression of viremia and clinical disease.

TABLE 3
Responses by HLA restriction of
CMVpp65-specific T-cells administered.

| | N | | | | |
|---|---|---|---|---|---|
| | TREATED | | | | |
| RESTRICTING | AND | | | | |
| HLA OF T- | EVAL- | RESPONSES | | | |
| CELLS (EPITOPE) | UABLE | CR | PR | SD | POD |
| HLA-A0201 | 19 | 12 | 2 | 3 | 2 |
| (NLVPMVATV) | | | | | |
| HLA-B0702 | 8 | 8 | 0 | 0 | 0 |
| (TPRVTGGGAM or | | | | | |
| RPHERNGFTV) | | | | | |
| HLA-B0801 | 3 | 3 | 0 | 0 | 0 |
| (DVEEDLTMT) | | | | | |
| HLA B4401-3/B4001 | 5 | 2 | 1 | 1 | 0 |
| (HERNGFTVL) | | | | | |
| HLA-B3501 (N = 3) | 7 | 0 | 0 | 0 | 7 |
| (IPSINVHHY) | | | | | |
| HLA-B3502 (N = 3) | | | | | |
| (QMQARLTVS) | | | | | |
| HLA-B3508 (N = 1) | | | | | |
| (EVQAIRETVE) | | | | | |
| HLA-A2601 | 3 | 0 | 0 | 0 | 3 |
| (INVHHYPSAA) | | | | | |

TABLE 4
Immunodominant HLA alleles detected in other
CMVpp65 specific T-cells administered

| HLA Allele | N | Response | | | |
|---|---|---|---|---|---|
| Restriction (Epitope) | Evaluable | CR | PR | SD | POD |
| A2407 | 1 | 0 | 0 | 0 | 1 |
| (QYDPVAALF) | | | | | |
| A2902 | 1 | 1 | 0 | 0 | 0 |
| (VCSMENTRAT) | | | | | |
| A3001 | 1 | 0 | 1 | 0 | 0 |
| (RVSQPSLIL) | | | | | |
| B0705 | 1 | 1 | 0 | 0 | 0 |
| (GVMTRGRLKA) | | | | | |
| B1801 | 1 | 0 | 1 | 0 | 0 |
| (KYQEFFWDAN) | | | | | |
| B2704 | 1 | 1 | 0 | 0 | 0 |
| (VSVNVHNPT) | | | | | |
| DRB1 0301 | 1 | 0 | 0 | 0 | 1 |
| (QPFMRPHERNG) | | | | | |
| DRB1 0701 | 1 | 0 | 0 | 0 | 1 |
| (SGKLFMHVTLG) | | | | | |
| DRB1 1101 | 1 | 0 | 1 | 0 | 0 |
| (FTSQYRIQGKL) | | | | | |

As can be seen, of 19 patients who received T-cells specific for a CMVpp65 epitope presented by HLA A201, 14 achieved a CR or PR. Of 9 treated with T-cells specific for an immunodominant epitope presented by HLA B0702, 8 achieved a CR. Similarly, immunodominant T-cells restricted by HLA A2402 (N=2) and B0801 (N=3) induced CRs in each of the 5 cases treated.

In contrast, 7/7 recipients of CMVpp65-specific T-cells specific for an immunodominant epitope presented by an allelic variant of HLA B35 failed to respond. Similarly, immunodominant T-cells specific for epitopes of A2601 (N=3), A2407 (N=1) and B5001 (N=1) failed to clear infection or reduce viremia.

These results provide evidence that immunodominant epitopes presented by specific HLA alleles induce T-cells that had better therapeutic activity in vivo, and thus these specific HLA alleles are immunodominant relative to the other HLA alleles.

The patients who received transplants from the donors who also agreed to have their CMVpp65-specific T-cells included in the bank for use in individuals other than to whom they also donated an HLA compatible HSCT were also retrospectively examined. The reason was that T-cell depleted transplants from such donors, which usually contain 2-8×10³ T-cells/Kg recipient weight, would also provide small numbers of immunodominant CMV-specific T-cells, since the frequency of IFNγ+ CMV specific T-cells in the blood in seropositive donors was in the range of 0.1-1% of the circulating T-cells. The results of this initial analysis are presented in Table 5.

TABLE 5
Analysis of CMV reactivation, disease and ultimate response to CMV-
directed therapy in patients who received transplants from HLA compatible donors who also
contributed cells for the bank as third party donors.

| | | Level of | | | | | | |
|---|---|---|---|---|---|---|---|---|
| | | [symbol] CMV Reactivation [symbol] | | | | | | |
| | | | Low | High | | | | |
| | | | (2-13/Slide | (≥100/slide | CMV | Rx T- | Type of | Ult. No |
| HLA Allele | N | No | ≤1000) | >slide) | Disease | Cells | Disease | Response |
| B0702 | 25 | 9 | 7 | 9 | 0 | 6 | — | 0 |
| A0201 | 36 | 14 | 12 | 10 | 1 | 7 | BAL + L.P. | 0 |
| No B0702 | 29 | 11 | 12 | 6 | 1 | — | BAL + L.P. | |
| B0801 | 16 | 8 | 4 | 4 | 1 | — | Other | 0 |
| No B0702 | | | | | | | | |
| B35 | 13 | 2 | 3 | 8 | 7 | — | 1 Meningoma | 6 |
| No A02 or B07 | | | | | | | 1 Hepatitis | |
| | | | | | | | 3 Pneumonia | |
| | | | | | | | 2 Other | |
| A1101 | 13 | 4 | 2 | 7 | 2 | — | 1 Pneumonia | 2 |
| No A02 or B7 | | | | | | | 1 Colitis | |
| A2601 | 9 | 3 | 3 | 3 | 3 | — | 2 Other | 1 |
| | | | | | | | 1 Pneumonia | |
| A0101 | 16 | 9 | 3 | 4 | 3 | — | 1 Meningoma | 2 |
| No A2 or B7 | | | | | | | 1 Other | |
| | | | | | | | 1 Pneumonia | |
| A2402 | 13 | 5 | 3 | 5 | 2 | — | 1 Pneumonia | 1 |
| No A2 or B7 | | | | | | | | |
| A0301 | 8 | 3 | 2 | 3 | 1 | — | 1 CSF+ | 0 |
| No A2 or B7 | | | | | | | | |

Again, as seen in the patients treated with CMVpp65-specific T-cells, recipients of transplants from donors sharing the HLA B0702 and A0201 alleles had a low risk of developing CMV disease, and virema consistently responded to treatment, while those who received grafts from donors lacking these alleles, had a significant incidence of overt infection. This was again particularly observed in patients bearing variants of HLA B35 who lacked either HLA B0702 or A0201.

The clinical data permitted us to determine a hierarchy of certain HLA alleles presenting immunodominant epitopes of CMVpp65 eliciting peptide-specific T cell responses (see Table 6), an example of a Representation of Activity.

TABLE 6
Hierarchy of HLA alleles presenting
immunodominant epitopes of CMVpp65
eliciting peptide-specific T cell responses.

| RESTRICTING HLA OF T CELL LINE | RANK^{a} |
|---|---|
| HLA-B0702 | 1 |
| HLA-A0201 | 2 |
| HLA-B0801 | 3 |
| HLA-B4401 | 4 |

^{a}A higher rank corresponds to greater clinical effectiveness of the T cell line restricted by the HLA allele in treatment of human patients having CMV infection or persistent viremia who failed to respond to antiviral drugs
Accordingly, HLA-A0201, HLA-B0801, and HLA-B4401 are subdominant HLA alleles.

### 6.3. Example 3: Combination Therapy with Anti-PD1 Antibody Based on the HLA Allele Hierarchy Established in Example 2

A patient presents with CMV infection. The patient is HLA-B0702⁺ and HLA-B0801⁺. According to the HLA hierarchy established in Example 2 and illustrated in Table 6, HLA-B0801 is a subdominant HLA allele relative to HLA-B0702 (i.e., the epitope presented by HLA-B0801 allele is a subdominant epitope relative to the epitope presented by HLA-B0702). A CTL line that is restricted by HLA-B0801 is selected and infused into the patient in combination with infusion of nivolumab. The patient is monitored before, during, and after the combination therapy for response by clinical assessments and quantitation of peripheral blood CMV DNA copy.

### 7. INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of treating a human patient having or suspected of having a pathogen or cancer, comprising:
(1) administering to the human patient an inhibitory immune checkpoint inhibitor; and
(2) administering to the human patient a population of human cells comprising antigen-specific T cells that are specific for an antigen of the pathogen or cancer and are derived from a T cell line restricted by a first HLA allele or HLA allele combination expressed by the diseased cells in the human patient;
wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen.

2. The method of claim 1, wherein the inhibitory immune checkpoint inhibitor is an inhibitor of Programmed Cell Death 1 (PD1), Programmed Death Ligand 1 (PD-L1), Programmed Death Ligand 1 (PD-L2), Cytotoxic T-Lymphocyte Associated Protein 4 (CTLA4), Lymphocyte Activating 3 (LAG3), T-Cell Immunoglobulin And Mucin Domain-Containing Protein 3 (TIM3), V-Domain Ig Suppressor Of T Cell Activation (VISTA), Adenosine A2a Receptor (A2aR), B7 Homolog 3 (B7-H3), B7 Homolog 4 (B7-H4), B and T lymphocyte associated (BTLA), Indoleamine 2,3-Dioxygenase (IDO), Tryptophan 2,3-Dioxygenase (TDO), or Killer-Cell Immunoglobulin-Like Receptor (KIR).

3. The method of claim 1 or 2, wherein the inhibitory immune checkpoint inhibitor is an antibody that binds to and inhibits the activity of the inhibitory immune checkpoint.

4. The method of claim 3, wherein the antibody is a monoclonal antibody.

5. The method of claim 2, wherein the inhibitory immune checkpoint inhibitor is an inhibitor of PD1.

6. The method of claim 5, wherein the inhibitory immune checkpoint inhibitor is a monoclonal antibody that binds to and inhibits the activity of PD1.

7. The method of claim 6, wherein the monoclonal antibody is nivolumab, pidilizumab, MEDI0680, or pembrolizumab.

8. The method of claim 2, wherein the inhibitory immune checkpoint inhibitor is an inhibitor of PD-L1.

9. The method of claim 8, wherein the inhibitory immune checkpoint inhibitor is a monoclonal antibody that binds to and inhibits the activity of PD-L1.

10. The method of claim 9, wherein the monoclonal antibody is mpd13280A, durvalumab, avelumab, bms-936559, or atezolizumab.

11. A method of treating a human patient having or suspected of having a pathogen or cancer, comprising:
(1) administering to the human patient a stimulatory immune checkpoint activator; and
(2) administering to the human patient a population of human cells comprising antigen-specific T cells that are specific for an antigen of the pathogen or cancer and are derived from a T cell line restricted by a first HLA allele or HLA allele combination expressed by the diseased cells in the human patient;
wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen.

12. The method of claim 11, wherein the stimulatory immune checkpoint activator is an activator of CD27, CD28, CD40, CD122, CD137, OX40, Glucocorticoid-Induced TNFR-Related Protein Ligand (GITR), or Inducible T-Cell Costimulator (ICOS).

13. The method of any of claims 1-12, wherein the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination based on recognition of the antigen.

14. The method of any of claims 1-13, wherein the first HLA allele or HLA allele combination is classified as a subdominant HLA allele or HLA allele combination based on being associated with an indication of relative activity based on recognition of the antigen that is lower than the relative activity associated with a second HLA allele or HLA allele combination expressed by the diseased cells according to a representation, which representation (i) identifies a plurality of HLA alleles and optionally HLA allele combinations, and (ii) discloses indications of relative activities of T cell lines, each recognizing at least one epitope of the antigen, and restricted by different ones of the HLA alleles or HLA allele combinations in the plurality; wherein in the representation each identified HLA allele or HLA allele combination is associated with the respective indication of relative activity of the T cell line restricted by the HLA allele or HLA allele combination, the relative activities being relative measures of known activity of the T cell lines based on recognition of the antigen.

15. The method of claim 14, wherein a T cell line restricted by the second HLA allele or HLA allele combination is available and suitable for therapeutic administration to the human patient.

16. A method of selecting a T cell line for therapeutic administration, in combination with administration of an inhibitory immune checkpoint inhibitor or a stimulatory immune checkpoint activator, to a human patient having or suspected of having a pathogen or cancer, comprising:
selecting a T cell line that recognizes at least one epitope of an antigen of the pathogen or cancer and is restricted by a first HLA allele or HLA allele combination expressed by the diseased cells in the human patient, wherein the first HLA allele or HLA allele combination is a subdominant HLA allele or HLA allele combination among HLA alleles and HLA allele combinations expressed by the diseased cells with respect to activity of T cells restricted by the respective HLA allele or HLA allele combination based on recognition of the antigen.

17. The method of claim 16, wherein the first HLA allele or HLA allele combination is a subdominant HLA allele or HLA allele combination among HLA alleles and HLA allele combinations expressed by the diseased cells with respect to activity of T cells that are suitable for therapeutic administration to the human patient and that are restricted by the respective HLA allele or HLA allele combination.

18. The method of claim 16 or 17, wherein the first HLA allele or HLA allele combination is classified as a subdominant HLA allele or HLA allele combination based on being associated with an indication of relative activity based on recognition of the antigen that is lower than the relative activity associated with a second HLA allele or HLA allele combination expressed by the diseased cells according to a representation, which representation (i) identifies a plurality of HLA alleles and optionally HLA allele combinations, and (ii) discloses indications of relative activities of T cell lines, each recognizing at least one epitope of the antigen, and restricted by different ones of the HLA alleles or HLA allele combinations in the plurality; wherein in the representation each identified HLA allele or HLA allele combination is associated with the respective indication of relative activity of the T cell line restricted by the HLA allele or HLA allele combination, the relative activities being relative measures of known activity of the T cell lines based on recognition of the antigen.

19. The method of claim 18, wherein a T cell line restricted by the second HLA allele or HLA allele combination is available and suitable for therapeutic administration to the human patient.

20. A method of selecting a T cell donor from whom to derive a T cell line for therapeutic administration, in combination with administration of an inhibitory immune checkpoint inhibitor or a stimulatory immune checkpoint activator, to a human patient having or suspected of having a pathogen or cancer, comprising:
selecting a T cell donor, using a first representation that (i) identifies a first plurality of HLA alleles and optionally HLA allele combinations, and (ii) discloses indications of relative frequencies of generation of T cell lines, each recognizing at least one epitope of an antigen of the pathogen or the cancer, and restricted by different ones of said HLA alleles or HLA allele combinations in the first plurality; wherein in the first representation each identified HLA allele or HLA allele combination is associated with the respective indication of relative frequency of generation of said T cell lines restricted by the respective HLA allele or HLA allele combination; wherein:
(A) the T cell donor selected has a first HLA allele or HLA allele combination in common with the diseased cells in the human patient that is associated in the first representation with an indication of the highest frequency of generation; and
(B) the first HLA allele or HLA allele combination of the selected T cell donor is a subdominant HLA allele or HLA allele combination among HLA alleles and HLA allele combinations expressed by the diseased cells with respect to activity of T cells restricted by the respective HLA allele or HLA allele combination based on recognition of the antigen.

21. The method of claim 20, wherein the first HLA allele or HLA allele combination is classified as a subdominant HLA allele or HLA allele combination based on being associated with an indication of relative activity that is lower than the relative activity associated with a second HLA allele or HLA allele combination of the diseased cells according to a second representation, which second representation (I) identifies a second plurality of HLA alleles and optionally HLA allele combinations, and (II) discloses indications of relative activities of T cell lines, each recognizing at least one epitope of an antigen, and restricted by different ones of the HLA alleles or HLA allele combinations in the second plurality; wherein in the second representation each identified HLA allele or HLA allele combination is associated with the respective indication of relative activity of the T cell line restricted by the HLA allele or HLA allele combination, the relative activities being relative measures of known activity of the T cell lines based on recognition of the antigen.

22. The method of claim 20 or 21, wherein the T cell donor is allogeneic to the human patient.

23. The method of claim 22, wherein the human patient has been the recipient of a transplant from a transplant donor, and the T cell donor is a third party donor that is different from the transplant donor.

24. A method of obtaining a T cell line for therapeutic administration, in combination with administration of an inhibitory immune checkpoint inhibitor or a stimulatory immune checkpoint activator, to a human patient having or suspected of having a pathogen or cancer, comprising:
(a) selecting a T cell donor according to the method of any of claims 20-23; and
(b) generating a T cell line from the selected T cell donor, which T cell line is restricted by the first HLA allele or HLA allele combination and recognizes at least one epitope of the antigen.

25. A method of treating a human patient having or suspected of having a pathogen or cancer, comprising:
(a) selecting a T cell line for therapeutic administration to the human patient according to the method of any of claims 16-19;
(b) administering to the human patient a population of human cells comprising antigen-specific T cells that are specific for the antigen and are derived from the selected T cell line; and
(c) administering to the human patient an inhibitory immune checkpoint inhibitor.

26. The method of claim 25, wherein the inhibitory immune checkpoint inhibitor is an inhibitor of PD1, PD-L1, PD-L2, CTLA-4, LAG3, TIM-3, VISTA, A2AR, B7-H3, B7-H4, BTLA, IDO, TDO, or KIR.

27. The method of claim 25 or 26, wherein the inhibitory immune checkpoint inhibitor is an antibody that binds to and inhibits the activity of the inhibitory immune checkpoint.

28. The method of claim 27, wherein the antibody is a monoclonal antibody.

29. The method of claim 25, wherein the inhibitory immune checkpoint inhibitor is an inhibitor of PD1.

30. The method of claim 29, wherein the inhibitory immune checkpoint inhibitor is a monoclonal antibody that binds to and inhibits the activity of PD1.

31. The method of claim 30, wherein the monoclonal antibody is nivolumab, pidilizumab, MEDI0680, or pembrolizumab.

32. The method of claim 25, wherein the inhibitory immune checkpoint inhibitor is an inhibitor of PD-L1.

33. The method of claim 32, wherein the inhibitory immune checkpoint inhibitor is a monoclonal antibody that binds to and inhibits the activity of PD-L1.

34. The method of claim 33, wherein the monoclonal antibody is mpd13280A, durvalumab, avelumab, bms-936559, or atezolizumab.

35. A method of treating a human patient having or suspected of having a pathogen or cancer, comprising:
(a) selecting a T cell line for therapeutic administration to the human patient according to the method of any of claims 16-19;
(b) administering to the human patient a population of human cells comprising antigen-specific T cells that are specific for the antigen and are derived from the selected T cell line; and
(c) administering to the human patient a stimulatory immune checkpoint activator.

36. The method of claim 35, wherein the stimulatory immune checkpoint activator is an activator of CD27, CD28, CD40, CD122, CD137, OX40, GITR, or ICOS.

37. The method of any of claims 1-19 and 25-36, wherein the T cell line is derived from a human donor that is allogeneic to the human patient.

38. The method of claim 37, wherein the human patient has been the recipient of a transplant from a transplant donor, and the human donor is a third party donor that is different from the transplant donor.

39. The method of any of claims 1-38, which further comprises a step of ascertaining the HLA assignment of the diseased cells in the human patient.

40. The method of claim 39, wherein the step of ascertaining comprises typing at least 4 HLA loci.

41. The method of any one of claims 1-19 and 25-40, wherein the activity of T cells is in vitro cytotoxic activity of the T cells against cells expressing the antigen.

42. The method of any one of claims 1-19 and 25-40, wherein the activity of T cells is in vivo clinical efficacies of the T cells in treatment of human patients having the pathogen or cancer.

43. The method of any one of claims 1-19 and 25-42, which further comprises a step of generating the T cell line restricted by the first HLA allele or HLA allele combination.

44. The method of claim 43, wherein the step of generating the T cell line restricted by the first HLA allele or HLA allele combination comprises ex vivo sensitizing T cells to the antigen.

45. The method of any one of claims 1-19 and 25-44, wherein the T cell line lacks substantial cytotoxicity in vitro toward antigen presenting cells that are not loaded or genetically engineered to express one or more peptides or proteins derived from the antigen.

46. The method of any of claims 1-45, wherein the antigen is an antigen of a pathogen.

47. The method of claim 46, wherein the pathogen is a virus, bacterium, fungus, helminth or protist.

48. The method of claim 47, wherein the pathogen is a virus.

49. The method of claim 48, wherein the virus is cytomegalovirus (CMV).

50. The method of claim 48, wherein the virus is Epstein-Barr virus (EBV).

51. The method of claim 48, wherein the virus is BK virus (BKV), John Cunningham virus (JCV), human herpesvirus, human papillomavirus (HPV), hepatitis B virus (HBV), hepatitis C virus (HCV), herpes simplex virus (HSV), varicella zoster virus (VZV), Merkel cell polyomavirus (MCV), adenovirus (ADV), human immunodeficiency virus (HIV), influenza virus, ebola virus, poxvirus, rhabdovirus, or paramyxovirus.

52. The method of any of claims 1-45, wherein the antigen is an antigen of a cancer.

53. The method of claim 52, wherein the antigen is Wilms Tumor 1 (WT1).

54. A pharmaceutical composition comprising:
(1) an inhibitory immune checkpoint inhibitor; and
(2) a population of human cells comprising antigen-specific T cells that are specific for an antigen of the pathogen or cancer and are derived from a T cell line restricted by a first HLA allele or HLA allele combination;
wherein the first HLA allele or HLA allele combination is a subdominant HLA allele or HLA allele combination with respect to activity of T cells restricted by the respective HLA allele or HLA allele combination based on recognition of the antigen.

55. The pharmaceutical composition of claim 54, wherein the inhibitory immune checkpoint inhibitor is an inhibitor of PD1, PD-L1, PD-L2, CTLA-4, LAG3, TIM-3, VISTA, A2AR, B7-H3, B7-H4, BTLA, IDO, TDO, or KIR.

56. A pharmaceutical composition comprising:
(1) a stimulatory immune checkpoint activator; and
(2) a population of human cells comprising antigen-specific T cells that are specific for an antigen of the pathogen or cancer and are derived from a T cell line restricted by a first HLA allele or HLA allele combination;
wherein the first HLA allele or HLA allele combination is a subdominant HLA allele or HLA allele combination with respect to activity of T cells restricted by the respective HLA allele or HLA allele combination based on recognition of the antigen.

57. The pharmaceutical composition of claim 56, wherein the stimulatory immune checkpoint activator is an activator of CD27, CD28, CD40, CD122, CD137, OX40, GITR, or ICOS.

58. The pharmaceutical composition of any of claims 54-57, wherein the first HLA allele or HLA allele combination is classified as a subdominant HLA allele or HLA allele combination based on being associated with an indication of lower activity based on recognition of the antigen that is lower than the relative activity associated with a second HLA allele or HLA allele combination according to a representation, which representation (i) identifies a plurality of HLA alleles and optionally HLA allele combinations, and (ii) discloses indications of relative activities of T cell lines, each recognizing at least one epitope of an antigen, and restricted by different ones of the HLA alleles or HLA allele combinations in the plurality; wherein in the representation each identified HLA allele or HLA allele combination is associated with the respective indication of relative activity of the T cell line restricted by the HLA allele or HLA allele combination, the relative activities being relative measures of known activity of the T cell lines based on recognition of the antigen.

59. The pharmaceutical composition of any of claims 54-58, wherein the antigen is an antigen of a pathogen.

60. The pharmaceutical composition of claim 59, wherein the pathogen is a virus, bacterium, fungus, helminth or protist.

61. The pharmaceutical composition of claim 60, wherein the pathogen is a virus.

62. The pharmaceutical composition of claim 61, wherein the virus is CMV.

63. The pharmaceutical composition of claim 61, wherein the virus is EBV.

64. The pharmaceutical composition of claim 61, wherein the virus is BKV, JCV, human herpesvirus, HPV, HBV, HCV, HSV, VZV, MCV, ADV, HIV, influenza virus, ebola virus, poxvirus, rhabdovirus, or paramyxovirus.

65. The pharmaceutical composition of any of claims 54-58, wherein the antigen is an antigen of a cancer.

66. The pharmaceutical composition of claim 65, wherein the antigen is WT1.

67. The pharmaceutical composition of any one of claims 54-66, wherein the T cell line lacks substantial cytotoxicity in vitro toward antigen presenting cells that are not loaded or genetically engineered to express one or more peptides or proteins derived from the antigen.
